SEQUENCE LISTING (7 sequences)

SEQ ID NO: 1 - DNA, 23 bp, Artificial Sequence
  Description of Artificial Sequence Primer
cctcccgctgttttgacctccta

SEQ ID NO: 2 - DNA, 23 bp, Artificial Sequence
  Description of Artificial Sequence Primer
atcccggcttcaacacgcatacc

SEQ ID NO: 3 - DNA, 24 bp, Artificial Sequence
  Description of Artificial Sequence Primer
gtgggccccttagcaatgtcttag

SEQ ID NO: 4 - DNA, 19 bp, Artificial Sequence
  Description of Artificial Sequence Primer
caccccgtttccccaatga

SEQ ID NO: 5 - protein, 6 aa, Artificial Sequence
  Description of Artificial Sequence Synthetic peptide
CXIXNN

SEQ ID NO: 6 - protein, 4 aa, Artificial Sequence
  Description of Artificial Sequence Synthetic peptide
LXHG

SEQ ID NO: 7 - protein, 10 aa, Artificial Sequence
  Description of Artificial Sequence Synthetic peptide
KPKLFFIQAC

(19) United States
(12) Patent Application Publication
(10) Pub. No.: US 2006/0057109 A1
(43) Pub. Date: Mar. 16, 2006

(54) Method of using anti-apoptotic factors in gene expression

(76) Inventors: David J. Waxman, Newton, MA (US); Pamela S. Schwartz, Seattle, WA (US)

Correspondence Address: RONALD I. EISENSTEIN, 100 SUMMER STREET, NIXON PEABODY LLP, BOSTON, MA, 02110, US

(21) Appl. No.: 10/509,533

(22) PCT Filed: Mar. 25, 2003
(86) PCT No.: PCT/US03/08743

§ 371 Date: May 26, 2005

Related U.S. Application Data
(60) Provisional application No. 60/367,311, filed on Mar. 25, 2002.

(51) Int. Cl. A61K 48/00; A61K 31/7072; A61K 31/66; A61K 31/522

(52) U.S. Cl. 424/93.1; 514/44; 514/109; 514/49; 514/263.32

(57) ABSTRACT

The present invention is directed to methods of prolonging the expression of a heterologous gene (transgene) in a cell, preferably a malignant cell. This method can be used to increase the concentration of a chemotherapeutic agent in a target cellular environment. Preferably, the present invention relates to methods of inhibiting apoptotic cell death to enhance transgene expression, such as gene-directed enzyme/prodrug therapy.

### GOVERNMENT SUPPORT

This invention was made with Government Support under Contract Number CA49248 awarded by the National Institutes of Health. The Government has certain rights in the invention.

### BACKGROUND OF INVENTION

1. Field of the Invention

The present invention is directed to methods of prolonging the expression of a heterologous gene (transgene) in a cell, preferably a malignant cell. This method can be used to increase the concentration of a chemotherapeutic agent in a target cellular environment. Preferably, the present invention relates to methods of inhibiting apoptotic cell death to enhance transgene expression, such as gene-directed enzyme/prodrug therapy.

2. Background

Significant attention has been directed to the expression of a heterologous gene in a cell. By appropriate selection of the gene, a number of different objectives can be achieved. For example, the gene can be used to express a desired enzyme to replace a damaged or inoperative native enzyme or supplement the cell's metabolic pathways. The gene can also be used to express a protein, which can, for instance, be an anti-angiogenic factor, an immune modulator, or a tumor suppressor, or can catalyze bioactivation of a chemotherapeutic prodrug. Thus, this approach can be used to accomplish a desired goal such as limiting tumor spread. In some instances, depending upon the cell and the protein that is expressed, the cell will be damaged, either directly or indirectly as a result of the expression. This can limit the cell's ability to express the protein at a level or for the duration required to achieve the desired result. This limitation can limit the effectiveness of the gene or the protein therapy. It can also necessitate repeating the process of transfecting the cell to re-introduce the heterologous gene. It would be desirable to limit this process, particularly where the cell is within an immune competent organism, because the means used to bring the gene to a given cell, such as a vector, can upon repeated use, cause an antigenic reaction to that means. It would therefore be desirable to have a method to extend the expression of a desired gene.

Conventional anti-cancer treatments, such as surgery and the use of cytotoxic chemotherapeutic drugs, have several major limitations. These include systemic, host tissue toxicity, which is often dose-limiting, and the emergence of subpopulations of drug-resistant tumor cells. Novel anti-cancer approaches using expression of a gene as a mode of treatment, aim to overcome these limitations.

Several novel cancer gene therapies have been introduced. These are aimed at oncogene inactivation, expression of an anti-angiogenic protein, correcting the loss of tumor suppressor genes, introduction of drug resistance genes into drug-sensitive host tissue such as bone marrow, enhancement of the anti-tumor immune response by immunotherapy using immune modulators such as cytokines and B7, activation of receptor-mediated cell death by expression of cytotoxic death receptor ligands, and the delivery to tumors of genes that code for enzymes that activate cancer chemotherapeutic prodrugs (Waxman et al., Drug Metab Rev. 31:503-522 (1999)). However, these and other gene therapy technologies are somewhat limited by their inability to deliver therapeutic genes to a population of tumor cells with high efficiency.

In contrast to tumor suppressor gene replacement and oncogene inactivation, gene-directed enzyme prodrug therapy (GDEPT) is not dependent on the genetic modification of each individual tumor cell, making this approach particularly promising as an anti-cancer therapy.

In GDEPT, sometimes referred to as prodrug activation, or suicide gene therapy, a gene therapy vector is used to deliver to tumor cells a transgene that encodes a prodrug-activating enzyme. Activation of a non-toxic, or substantially non-toxic prodrug to create a more toxic drug metabolite leads to the killing of tumor cells in which the metabolite is produced.

The effectiveness of the GDEPT strategy can be greatly enhanced, however, by using drugs that exhibit a “bystander effect” (Pope et al., Eur J Cancer 33:1005-1016 (1997)). Bystander cytotoxicity results when active drug metabolites diffuse or are otherwise transferred from their site of generation within a transduced tumor cell to a neighboring, naive tumor cell. Ideally, the bystander effect leads to significant tumor regression even when a minority of tumor cells is transduced with the prodrug activation gene (e.g., Chen et al., Hum Gene Ther. 6:1467-1476 (1995); Freeman et al., Cancer Res. 53:5274-5283 (1993)). Bystander cytotoxic responses may also be mediated through the immune system, following its stimulation by interleukins and other cytokines secreted by tumor cells undergoing cell death (Gagandeep et al., Cancer Gene Ther. 3:83-88 (1996)). Bystander effects are also associated with approaches that lead to the expression or production by the target cell of a soluble or secretable factor. Examples include factors with angiogenic, anti-angiogenic, cytotoxic or immune modulatory activity.

More recently, a prodrug activation/gene therapy strategy has been developed based on the use of a cytochrome P450 gene (“CYP” or “P450”) in combination with a cancer chemotherapeutic agent that is activated through a P450-catalyzed monooxygenase reaction (Chen and Waxman, Cancer Research 55:581-589 (1995); Wei et al., Hum. Gene Ther. 5:969-978 (1994); U.S. Pat. No. 5,688,773; U.S. Pat. No. 6,207,648). Unlike many other prodrug activation strategies, the P450-based drug activation strategy can utilize a mammalian prodrug activation gene (rather than a bacterially or virally derived gene), and also can be carried out using chemotherapeutic drugs that are established and widely used in cancer therapy. Investigational anti-cancer prodrugs, and novel prodrugs designed to be activated by way of P450 metabolism can also be used in this therapy.

Gene therapy using a P450 gene is characterized by several important features: 1) there is an intrinsic differential between the therapeutic activity of the prodrug and that of the P450-activated drug metabolite; 2) it can substantially increase the concentration of activated drug in, or in the vicinity of a tumor cell, particularly when combined with localized delivery of the prodrug using a slow release polymer (Ichikawa et al., Cancer Res. 61:864-868 (2001)); and 3) it generates a bystander effect. However, the P450 enzyme system is not ideal because 1) P450 enzymes, in general, metabolize drugs and other foreign chemicals, including cancer chemotherapeutic drugs, at low rates, with a typical P450 turnover number (moles of metabolite formed/mole P450 enzyme) of only 10-50 per minute; and (2) P450 enzymes metabolize many chemotherapeutic drugs with a high Km value, typically in the millimolar range. This compares to plasma drug concentrations that are much lower, typically in the micromolar range for many chemotherapeutic drugs, including the anti-cancer P450 prodrugs cyclophosphamide (CPA) and ifosfamide. Thus, current approaches to P450 gene therapy may result in intratumoral prodrug activation at a low absolute rate and under conditions that are not saturating with respect to the prodrug substrate. Furthermore, since P450 is expressed at a very high level in liver tissue, most of the prodrug is metabolized in the liver, and only a small fraction of the administered chemotherapeutic prodrug is metabolized by the tumor cell P450 gene product using the currently available methods for P450 gene therapy (Chen and Waxman, Cancer Res. 55:581-589 (1995)). Therefore, although gene-based therapies such as the P450/prodrug activation system have shown good activity against many tumor types, further enhancement of the activity of this gene therapy would be desirable to increase the occurrence of clinically effective responses in cancer patients.

Several methods are known to enhance the chemosensitivity of tumor cells that are transduced with a prodrug activating P450 enzyme. In the case of CPA and other alkylating agents, enhanced cytotoxicity can be achieved using pharmacological approaches, including depletion of protective cellular small molecules, such as glutathione (GSH), or by decreasing the expression or activity of protective enzymes, such as glutathione S-transferases (GSTs) (Chen and Waxman, Biochem Pharmacol. 47:1079-1087 (1994); Ozols et al., Biochem Pharmacol. 36:147-153 (1987); Waxman, Cancer Res. 50:6449-6454 (1990)) or aldehyde dehydrogenases. Overexpression of GST enzymes can contribute to resistance of tumor cells to a chemically activated form of CPA, 4-hydroperoxy-CPA (4HC), and modulators that target the GSH/GST system may be useful in sensitizing tumor cells to CPA (Chen and Waxman, Cancer Res. 55:581-589 (1995)). However, it is herein shown that the chemosensitization of tumor cells to CPA by depletion of GSH is accompanied by a decreased ability of the P450-expressing tumor cells to generate cytotoxic CPA metabolites, and hence, limitation of the ability of these cells to confer a strong bystander cytotoxic effect. Therefore, it would be desirable to develop methods to increase P450 activity in a way that leads to an increase in the concentration and/or time of exposure to the active chemotherapeutic drug in the target cellular environment.

Several chemotherapeutic agents and other cytotoxic drugs have been shown to kill tumor cells by activating the mitochondrial/caspase 9 pathway of cell death (Green, 1998; Reed, 1999). Examples include etoposide, staurosporine, betulinic acid and CPA (Fulda et al., Cancer Res. 58:4453-4460 (1998); Schwartz and Waxman, Mol Pharmacol. 60:1268-1279 (2001); Sun et al., J Biol. Chem. 274:5053-5060(1999)).

Also, chemotherapeutic drug-induced DNA damage, such as that induced by CPA, has been shown to lead to the induction of apoptosis. DNA damage is sensed by a number of enzymes from the PI(3)-kinase family including ATM (ataxia telangiectasia mutated), DNA-PK (DNA-dependent protein kinase) and ATM (ataxia telangiectasia Rad3 related). Once the DNA damage is detected, these kinases initiate a phosphorylation cascade that involves cell cycle checkpoint, DNA repair proteins, and the induction of apoptosis (Ferri and Kroemer, Nat Cell Biol. 3:E255-263 (2001); Rich et al., Nature. 407:777-783 (2000)). Downstream proteins that have been shown to play critical roles in initiating DNA damage-induced apoptosis include the transcription factors p53 (Meek, Oncogene. 18:7666-7675 (1999)) and E2F-1 (Blattner et al., Mol Cell Biol. 19:3704-3713 (1999)). ATM and other DNA damage recognition molecules can activate a pathway that leads to p53 phosphorylation, thereby altering p53's transcriptional activity and increasing its stability (Meek, Oncogene. 18:7666-7675 (1999); Rich et al., Nature. 407:777-783 (2000)).

Several apoptotic molecules that contribute to the mitochondrial/caspase 9 apoptotic pathway are regulated by p53. Examples include the up-regulation of the pro-apoptotic factor Bax and the down regulation of the anti-apoptotic factor Bcl-2 when p53 becomes activated (Findley et al., Blood. 89:2986-2993 (1997); Perego et al., Cancer Res. 56:556-562 (1996)). Thus, p53 can link the detection of DNA damage induced by chemotherapeutic drugs to the induction of mitochondrial regulated apoptosis. The transcription factor E2F-1 has also been shown to become stabilized following DNA damage in a manner similar to p53 (Blattner et al, Mol Cell Biol. 19:3704-3713 (1999)). E2F-1 can activate an apoptotic response in the absence of p53 suggesting that it may be important in initiating DNA damage induced apoptosis in tumor cells containing mutated p53 (Lissy et al., Nature. 407:642-645 (2000)).

While some anti-cancer drugs induce a mitochondrial pathway of cell death, it has been demonstrated that anticancer drugs can also activate a cell surface death receptor/caspase 8 dependent pathway of cell death. Drugs that have been shown to induce this latter pathway include doxorubicin (Fulda et al, Cancer Res. S8:44534460 (1998)), the prodrug 5-fluorocytosine (5-FC) when activated to 5-fluorouracil (5-FU) by the enzyme cytosine deaminase (CD) (Tillman et al., Clin Cancer Res. 5:425-430 (1999)), cisplatin (Seki et al., Cancer Chemother Pharmacol. 45:199-206 (2000)), and the prodrug ganciclovir (GCV) when activated to a cytotoxic nucleoside triphosphate by herpes simplex virus thymidine kinase (HSV-tk) (Beltinger et al., Proc Natl Acad Sci U.S.A. 96:8699-8704 (1999)). Several mechanisms can explain this caspase 8-dependent response of tumor cells to cancer chemotherapeutic drugs.

One mechanism is based on the observation that chemotherapeutic drugs enhance the expression of Fas ligand and can stimulate a p53-dependent increase in cell surface expression of Fas, the receptor protein for Fas ligand (Friesen et al., Nat Med. 2:574-577 (1996); Muller et al., J Clin Invest. 99:403-413 (1997)), and the Trail death receptor DR5 (Wu et al., Oncogene. 18:6411-6418 (1999)) in certain tumor cell types. This leads to the killing of tumor cells in an autocrine or paracrine fashion (Mow et al., Curr Opin Oncol. 13:453462 (2001); Petak and Houghton, Pathol Oncol Res. 7:95-106 (2001); Petak et al., Cancer Res. 60:2643-2650 (2000)). These findings are supported by the observations that 5-fluorouracil treatment of thymidylate synthase-deficient colon carcinoma cells induces cytotoxicity that can be blocked by anti-Fas antibodies. This mechanism of cell death is thus dependent on Fas expression (Mow et al., Curr Opin Oncol. 13:453-462 (2001); Petak and Houghton, Pathol Oncol Res. 7:95-106 (2001)).

A second mechanism is based on the finding that cisplatin, doxorubicin, etoposide and ganciclovir, when activated by herpes simplex virus thymidine kinase, induce a ligand-independent caspase 8 pathway of cell death that is mediated by a FADD (Fas-associated death domain) dependent aggregation of the cell death receptor (Micheau et al., J Biol. Chem. 274:7987-7992 (1999); Beltinger et al, PNAS 96:8699-8704)).

Several novel anti-cancer therapies have recently been designed that specifically target factors in the apoptotic pathway. The major goal of these strategies is to modulate apoptotic pathway factors in a manner that leads to an increase in apoptotic cell death. Examples include targeting the mitochondrial cell death pathway by overexpression of pro-apoptotic proteins such as Bax (Kagawa et al., Cancer Res. 60:1157-1161 (2000)) or by targeting and down-regulating anti-apoptotic proteins such as Bcl-2 (Klasa et al., Clin Cancer Res. 6:2492-2500 (2000); Waters et al., J Clin Oncol. 18:1812-1823 (2000)). Other approaches, such as the treatment with cell death receptor ligands such as Trail (Tumor necrosis factor-related apoptosis-inducing ligand) (Ashkenazi et al., J Clin Invest. 104:155-162 (1999)) or the inhibition of IAPs (inhibitors of apoptosis) such as Survivin (Mesri et al., J Clin Invest. 108:981-990 (2001)), are designed to increase the activity of the receptor-mediated pathway of cell death (Ashkenazi et al., J Clin Invest. 104:155-162 (1999); Kagawa et al., Cancer Res. 61:3330-3338 (2001)). Additional approaches, which target both major cellular pathways of apoptosis, include delivery of a caspase gene or the expression of p53, which have shown promise in preclinical and in phase I clinical trials (Marcelli et al., Cancer Res. 59:382-390 (1999); Yamabe et al., Gene Ther. 6:1952-1959 (1999)).

Elucidation of the pathway of cell death that is induced by a particular chemotherapeutic drug or anti-cancer treatment can aid in the design of novel combination anti-cancer therapies. For example, CPA is known to induce a mitochondrial-mediated cell death pathway (Schwartz and Waxman, Mol Pharmacol. 60:1268-1279 (2001)), while Bcl-2 is known to block the mitochondrial apoptotic pathway. Therefore, enhanced chemosensitization to CPA may be achieved in the case of tumors that express Bcl-2 by using therapies that decrease expression or abolish the activity of the anti-apoptotic factor Bcl-2.

Therapies that have been proposed to be useful in augmenting the anti-cancer activity of drugs like CPA, include antisense oligonucleotides that target Bcl-2 (Reed et al., Cancer Res. 50:6565-6570 (1990); Ziegler et al., J Natl Cancer Inst. 89:1027-1036 (1997)) and intracellular expression of anti-Bcl-2 antibodies (Piche et al., Cancer Res. 58:2134-2140 (1998)). Similarly, expression of the pro-apoptotic factor Bax can be employed to counter the chemoresistant effects of Bcl-2 (Kagawa et al., Cancer Res. 60:1157-1161 (2000)) (Oltvai et al., Cell. 74:609-619 (1993)). Other anti-sense strategies can be designed to inhibit the expression of proteins belonging to the IAP family of caspase inhibitors, widely believed to be useful therapeutic targets for inhibition when treating proliferative diseases such as cancer (Korneluk et al., U.S. Pat. No. 6,300,492).

Other studies indicate that the simultaneous or sequential activation of the alternative, receptor-mediated apoptotic pathway may augment the chemotherapeutic effects of anti-cancer drugs. Receptor-mediated cell death can occur in tumor cells classified as type I cells even in the presence of high levels of Bcl-2, and as such, drugs and death receptor ligands that activate death receptor pathways can be used to kill tumor cells that are otherwise chemoresistant by virtue of Bcl-2 overexpression. One promising strategy for inducing receptor-mediated cell death uses the death receptor ligand Trail. Recombinant Trail can induce tumor regression with little systemic toxicity to healthy tissues, which are protected by the expression of decoy death receptors, which are down-regulated in many tumor cells but not in host tissues (Ashkenazi et al., J Clin Invest. 104:155-162 (1999); Kagawa et al., Cancer Res. 61:3330-3338 (2001); Rieger et al., FEBS Lett. 427:124-128 (1998)). Additionally, infection of tumor cells with adenovirus vectors engineered to express Trail leads to apoptosis of tumor cells but not normal cells. Moreover, expression of Trail confers bystander toxicity (Kagawa et al., Cancer Res. 61:3330-3338 (2001)).

Although apoptosis-inducing genes or other chemosensitization approaches can thus be used to induce or augment an anti-cancer response, the use of such pro-apoptotic factors as modulatory factors in the context of gene therapy using a prodrug-activating enzyme, or a soluble, or secretable, cytotoxic factor, poses a general dilemma identified herein: any modulation strategy that increases the chemosensitivity of the target tumor cell is also likely to undermine the effectiveness of the gene therapy by shortening the lifespan of the tumor cells that express the foreign gene, thereby decreasing the net production and release of active drug metabolites, or of soluble, or secretable, cytotoxic factor, into the surrounding tumor milieu. On the other hand, any effort to block the death of those cells that express the prodrug-activating enzyme or therapeutic factor presents the risk of generating an aggressive, drug-resistant tumor.

For example, attempts to further increase the killing of tumor cells by enhancing the chemosensitivity of P450-expressing tumor cells to CPA by chemical means (depletion of glutathione by treatment with buthionine sulfoximine) or by coexpression of pro-apoptotic factors, while resulting in the desired increase in chemosensitivity, is herein shown to be accompanied by accelerated killing of the tumor cells transduced with the prodrug-activating P450 gene. Consequently, the net production and release to the medium of bystander cytotoxic drug metabolites is decreased. P450-expressing tumor cells are further shown to lose functional P450 activity during the course of prodrug treatment, limiting the potential of these cells to generate activated prodrug metabolites.

Therefore, it would be desirable to develop a method to increase the net production over time of a cytotoxic prodrug metabolite or a soluble, or secretable, cytotoxic factor in the tumor milieu without shortening the life span of the factory cell that produces the chemotherapeutic agent or factor and also with decreased risk of generating a drug-resistant tumor.

### SUMMARY OF THE INVENTION

We have now discovered a method of increasing the production of a chemotherapeutic drug, or a secretable, therapeutic factor in, or in the vicinity of, a cell by selectively increasing the life span of the cell by inhibiting apoptosis.

The method is based upon the finding that the inhibition of apoptosis can lead to increased life span of the target cell while still allowing an alternative, irreversible cell death to occur. Thus, one can increase production of a desired therapeutic factor, such as a chemotherapeutic prodrug metabolite or a soluble, or secretable, therapeutic factor, by prolonging expression of a therapeutic gene and yet leave the cell susceptible to die more slowly via a different pathway.

In one embodiment, the invention provides a method of increasing the concentration of an active chemotherapeutic drug in, or in the vicinity of, a neoplastic cell infected with a vector comprising a nucleic acid encoding a prodrug activating enzyme comprising delivering to the neoplastic cell a vector encoding an apoptosis inhibiting agent. The “apoptosis inhibiting agent” (“AIA”), is also sometimes referred to herein as “IAP” (inhibitor of apoptosis) and is preferably a caspase pathway inhibiting agent. Caspase pathway inhibiting agents include, but are not limited to a caspase inhibitor, an anti-apoptotic Bcl-2 family member, or a death receptor pathway inhibitory agent. Preferably the caspase pathway inhibiting agent is a caspase inhibitor preferably p35, p49, CrmA, CiIP, OpIAP/CpIAP/AcIAP, ASFIAP (from viral sources), XIAP, hIAP1, hIAP2, Naip, Bruce, Survivin, and pIAP (from human or other mammalian sources) and DIAP1, DIAP2 (fly), CeIAP1, CeIAP2 (nematode) and SpIAP and ScIAP (yeast). Most preferably the IAP is p35 or Survivin. The prodrug activating enzyme is preferably a cytochrome P450. The term “IAP” is used herein and throughout the specification to refer to all anti-apoptotic factors, including factors than do not show sequence homology to XIAP or other established protein members of the conventional IAP family, as well as factors that inhibit death receptor-mediated apoptotic cell death, including FLIPs (Fas-associated death domain-like ice inhibitory proteins), decoy receptors and dominant negative FADD (Fas-associated death domain protein). The terms AIA and IAP, as used herein, also refer to any other means that inhibit apoptosis including proteins, small molecules, antisense oligonucleic acids and small inhibitory RNAs (siRNA) designed to target proteins or nucleic acids essential for the apoptotic cell death pathway.

In another embodiment, the invention provides a method of increasing the expression of a prodrug activating enzyme and hence the concentration of active chemotherapeutic drug in, or in the vicinity of, a target cell or tissue in a mammal in need thereof comprising the steps of providing a first vector comprising a nucleic acid encoding a prodrug activating enzyme and a second vector comprising a nucleic acid encoding an apoptosis inhibiting agent and subjecting the mammal to an appropriate prodrug. The target cell is preferably a neoplastic cell or a tumor-associated endothelial cell. The apoptosis inhibitory agent is preferably a caspase inhibitor, an anti-apoptotic Bcl-2 family member, a FLIP protein, a death receptor decoy protein or a dominant-negative FADD.

In a preferred embodiment, the invention provides a method of increasing the concentration of a chemotherapeutic drug in, or in the vicinity of, a cell or tissue in a mammal in need thereof comprising the steps of administering an AIA such as by providing a vector comprising a nucleic acid encoding a prodrug activating enzyme and a nucleic acid encoding an apoptosis inhibiting agent and subjecting the mammal to a prodrug that is activated by the prodrug activating enzyme. In a preferred embodiment, the vector further comprises nucleic acid encoding an LAP-inactivating factor or other pro-apoptotic factor, preferably Smac/Diablo, a caspase, p53, Bax, Bak, Bcl-Xs, Bad, Bik, Bid, apoptosis-inducing factor, or anti-sense IAP, or a small inhibitory RNA (siRNA), directed against (‘targeting’) the apoptosis inhibiting agent, an endogenous cellular IAP or other anti-apoptotic factor under control of a regulatable promoter.

In yet another embodiment, the invention provides a method of increasing the concentration of a chemotherapeutic drug in, or in the vicinity of, a cell or tissue in a mammal in need thereof comprising transducing the cells with a vector comprising a nucleic acid encoding a prodrug activating enzyme and simultaneously, before or after transducing the cell with said vector, providing the cell with an anti-apoptotic agent directly delivering a protein, such as an antibody or a soluble receptor decoy, a small molecule, a single- or double-stranded nucleic acid such as an antisense oligonucleotide or an siRNA. The key is that such agents inhibit apoptosis.

The phrase “appropriate prodrug” as used herein refers to a prodrug which corresponds to the prodrug activating enzyme that the cell is transduced with. Such prodrug-prodrug activating enzyme combinations are known to one skilled in the art and are exemplified in more detail below.

In yet another embodiment, the invention provides a method of increasing the expression and hence the concentration of a soluble, or secretable, therapeutic agent by transducing the cell with a vector encoding the therapeutic agent and providing the cell with an anti-apoptotic agent. This can be accomplished by known means, for example either by transferring a nucleic acid encoding the anti-apoptotic agent to the cell or providing the anti-apoptotic agent directly to the cell. Preferably the target cell is a neoplastic cell or a tumor-associated endothelial cell. Preferably, the agent is one that has anti-angiogenic, cytotoxic or immune modulatory activity. More preferably the therapeutic agent is endostatin, angiostatin, thrombospondin-1, VEGF (vascular endothelial growth factor) antibody, VEGF receptor-derived ectodomain, tumstatin and other integrin-binding or integrin-inhibiting molecules, 16 kd prolactin fragment, platelet factor 4, an antibody or an anti-sense or siRNA agent directed against an angiogenic factor, a tumor necrosis factor superfamily member, preferably TNFα, Fas ligand and Trail, and a cytokine or immune modulator, preferably interferon α, interferon β and interleukins 2, 12 and 18. Because this method enhances the concentration of the agent it does so in the target cell and/or in its vicinity.

In another embodiment, the invention provides a method for increasing vector spread in a host using an apoptosis inhibiting agent to suppress apoptosis. This results in greater vector spread and expression of the desired gene or gene product. The method can be done by known means. For example, administering an apoptosis inhibiting agent using a replicating vector. Alternatively, the apoptosis inhibiting agent is delivered via a non-replicating vector, which is administered in combination with (either simultaneous with, before or after) a replicating vector. Preferably, the apoptosis inhibiting agent is a caspase pathway inhibitor, most preferably p35. In a preferred embodiment, the replicating vector is a virus, preferably a tumor cell-replicating viral vector, most preferably an adenovirus. Preferably, the vector is one that codes for a prodrug-activating enzyme or a soluble, or secretable, therapeutic factor.

### BRIEF DESCRIPTION OF THE FIGURES

FIGS. 1A-1B show that glutathione depletion enhances CPA-induced cytotoxicity and decreases CPA 4-hydroxylase activity in 9L/P450 cells. FIG. 1A shows 9L/P450 cells which were either untreated or were pretreated with 50 μM BSO for 24 hr. Cells were then treated with 1 mM CPA, beginning at time 0, for times up to 72 hr, as indicated. Duplicate samples were stained with crystal violet (A₅₉₅) at each time point to quantitate relative cell protein content (‘cell survival’). Mean values (±range) were graphed. The 24 hr BSO pretreatment had no effect on cell growth, as seen by the coincidence of A₅₉₅ values at time 0. FIG. 1B shows 9L/P450 cells pretreated with BSO, as in panel A, which were treated with 1 mM CPA for times up to 28 hr. Cellular CPA 4-hydroxylase activity was assayed by incubating the cells in fresh media containing 1 mM CPA and 5 mM semicarbazide, added to stabilize the 4-OH-CPA metabolite, for the 4 hr time interval indicated at the bottom of panel B, left side. An aliquot (0.5 ml) of media was removed, derivatized, and then analyzed for 4-OH-CPA. Shown are the measured culture media concentrations of 4-OH-CPA (mean+/−range for n=2) (left panel). Shown on the right are crystal violet values, indicative of relative cell protein content of each sample.

FIGS. 2A-2C demonstrate IAP expression in 9L/P450 cells. 9L/P450 cells were infected with retrovirus encoding the IAPs p35 or Survivin. IAP expression was verified by reverse transcriptase PCR (FIG. 1A). 9L/P450 and 9L/P450-derived IAP cell lines were treated with 1 mM CPA and assayed for caspase 8 or caspase 9 activity. Shown in FIG. 1B are the caspase 8 and caspase 9 activities of the individual cell lines (mean+/−half the range for n=2 separate experiments). Additionally, 9L/P450 and 9L/P450/p35 cells were treated with 1 mM CPA for 72 hr and stained with crystal violet at each time point to quantitate cell survival (FIG. 1C) (mean+/−half the range, n=2).

FIG. 3A-3B show 9L/P450 cells that express p35 maintain CPA 4-hydroxylase activity when treated with CPA for 8 hr but not when treated with CPA continuously. 9L/P450 and 9L/P450/p35 cells were treated with 1 mM CPA continuously (FIG. 3A) or were treated with 1 mM CPA for 8 hr and then incubated in drug-free media for the duration of the experiment. Cellular CPA 4-hydroxylase activity at each time point (left side of each panel) was assayed as described in FIGS. 1A-C. Relative cell protein content (‘cell survival’) was determined by crystal violet staining at the time points indicated (A₅₉₅, middle). Cellular CPA 4-hydroxylase activity normalized to total cell protein was calculated by dividing the values shown on the left panel by the A₅₉₅ values shown in the middle panel (nmol 4-OH-CPA per ml media per A₅₉₅) (right).

FIGS. 4A-4D show characterization of clonal 9L/P450/p35 cell lines. Four clones of 9L/P450/p35 cells were generated by clonal selection of a pool of 9L/P450/p35 cells prepared by retroviral infection as described in FIG. 2A-2C. Total RNA isolated from the parental 9L/P450 cells (WT) and from the four 9L/P450/p35 clonal cell lines was amplified by RT-PCR using p35-specific primers. The 561 base pair cDNA product obtained in samples prepared either with (+RT) or without (−RT) reverse transcriptase was analyzed by agarose gel electrophoresis (FIG. 4A). Caspase 3 activity was assayed for each cell line following no drug treatment or treatment with 1 mM CPA for 48 hr (mean+/−half the range, n=2 separate experiments) (FIG. 4B). P450 2B6 protein content of the 9L/P450 cells and 9L/P450/p35 clonal cell lines was determined by Western blot analysis (20 μg total cell extract protein/lane) (FIG. 4C). Total cell extract from each cell line was assayed for P450 reductase activity by monitoring the reduction of cytochrome C (mean+/−half the range, n=2) (FIG. 4D).

FIGS. 5A-5C demonstrate that cells that express high levels of p35 retain high levels of CPA 4-hydroxylase activity following 8 hr CPA treatment. 9L/P450 cells and four 9L/P450/p35 clonal cell lines (p35-3, p35-7, p35-8 and p35-9) were untreated (FIG. 5A), were treated with 1 mM CPA continuously (FIG. 5B) or were treated with 1 mM CPA for 8 hr and then cultured in drug-free media for the duration of the experiment (FIG. 5C). At the times indicated each cell line was assayed for: cellular CPA 4-hydroxylase activity, as described in FIG. 1 (left set of graphs in each panel); relative cell growth rates determined by crystal violet staining (A₅₉₅ values, middle set of graphs), and 4-OH-CPA production per cell protein, calculated as described in FIGS. 3A-3B (right set of graphs). Data shown are mean+half the range values (n=2 samples) based on a representative experiment.

FIGS. 6A-6D demonstrate enhanced bystander cytotoxicity of 9L/P450/p35 tumor cells. The cytotoxicity conferred by 9L/P450 and 9L/P450/p35-9 cells on 9L/LacZ bystander cells was assayed using one of two protocols. Cells were cultured such that the 9L/LacZ cells were exposed to the second of two 8 hr CPA treatments given to the P450-containing 9L cells. Following this drug treatment cell survival was determined for each of the three cell lines (9L/P450, 9L/P450/p35-9 and 9L/LacZ) using a colony formation assay. In the experiment shown in FIGS. 6A and 6B (protocol 1) the 9L/LacZ bystander cells were plated on top of the 9L/P450 or 9L/P450/p35-9 cells, enabling the two cell populations to be in direct cell-cell contact. In the experiment shown in FIGS. 6C and 6D (protocol 2) the P450-containing cells were plated on cell culture inserts such that the co-cultured cell lines shared the same media but did not come in direct contact with each other. FIGS. 6A and 6C present the relative colony formation activity of the 9L/LacZ cells co-cultured with 9L/P450 cells, while FIGS. 6B and 6D present the relative colony formation activity of the 9L/LacZ cells co-cultured with 9L/P450/p35-9 cells. Colony formation in the absence of drug treatment was set=100% for each sample. Error bars represent mean±SD for n=4 replicates.

FIGS. 7A-7D show the time course for killing of 9L/P450 and 9L/P450/p35 cells by CPA. 9L/P450 and 9L/P450/p35-9 cells were untreated (FIG. 7A), were treated with 1 mM CPA continuously (FIG. 7B), or were given a single 8 hr treatment (FIG. 7C) or three 8 hr treatments (FIG. 7D) with 1 mM CPA. Cells were then cultured for up to 17 days following the initial drug treatment. Downward arrows in FIGS. 7C and 7D indicate the times at which each 8 hr CPA treatment was initiated. Cells remaining on the plates at each indicated time point were stained with crystal violet. Data shown are mean+/−half the range for A₅₉₅ values n=2 samples at each time point.

FIGS. 8A-8C show that 9L/P450/p35-9 cells loose CPA 4-hydroxylase activity following two 8 hr CPA treatments. CPA 4-hydroxylase activity of 9L/P450 and 9L/P450/p35-9 cells was assayed as described in FIG. 1 in cultures treated with 1 mM CPA given as a single 8 hr exposure (FIG. 8A), continuously (FIG. 8B) or for two 8 hr exposures spaced 3 days apart (FIG. 8C). Downward arrows indicate the times when the 8 hr CPA treatments were applied. Data shown are mean+/−half the range A₅₉₅ values for n=0.2 samples at each time point.

FIG. 9 shows that replicating adenovirus, which is deleted in the adenoviral E1b-55 kd gene, enhances expression of Adeno-βgal, a replication defective adenovirus that codes for β-galactosidase, to a ˜3-4-fold greater extent in 9L/P450/p35 cells compared to 9L/P450 cells that do not express p35, 9L/P450 and 9L/P450/p35 clone 9 cells, as in FIG. 5, were infected with Adeno β-gal (multiplicity of infection (MOI)=30) either alone, or in combination with E1b-55 kd-deleted adenovirus at an MOI of 0, 0.5, 1.5, 5 or 10, as indicated. Seven days later the cells were stained with X-gal, which marks the Adeno β-gal-infected cells blue. X-gal staining was quantitated by absorbance at 620 nm (A₆₂₀) and the results normalized to total cell number, determined by crystal violet staining (A₅₉₅). Data are presented as normalized A₆₂₀/A₅₉₅ ratios, mean±range for n=2 independent samples at each MOI of virus.

### DETAILED DESCRIPTION OF THE INVENTION

The present invention is directed to a method of increasing the expression of a desired gene or gene product in a target cell, preferably a neoplastic cell or a tumor-associated endothelial cell. The gene can be any type, such as one that encodes a prodrug-activating enzyme or one that provides for the production of a soluble or secretable therapeutic product, such as an anti-angiogenic, cytotoxic or immune modulatory factor (‘therapeutic gene’). By increasing the expression of such a gene or gene product, one can increase the concentration of an active chemotherapeutic agent in the cellular environment. We have discovered that delivery of an anti-apoptotic agent to a cell containing such a therapeutic gene (the “therapeutic factory cell”) increases the life span of the cell and increases the net formation by the factory cell of an active chemotherapeutic agent capable of killing neighboring cells and eventually also the transduced factory cell. One preferred method of having that cell contain the therapeutic gene is by infecting that cell with a vector. However, any other known method can be used for example gene gun, liposomes, catheters, etc.

The method is based upon the finding that a selective inhibition of apoptosis can lead to an increased life span of the target cell allowing the cell to continue to express the transgene for a longer period of time. We have further discovered that inhibition of the apoptotic pathway slows down but ultimately does not prevent the factory cell from dying. Thus, one can convert more of a prodrug into an active chemotherapeutic drug, and one can produce and/or secrete more of a therapeutic product over time, while leaving the cell susceptible to destruction by a different pathway.

Without wishing to be bound by a theory, we propose that the method of the present invention to use apoptosis inhibiting agents to increase the lifespan of a cell expressing a prodrug activating enzyme or a soluble or secretable therapeutic factor has several advantages in treating disease, most notably neoplastic disease. First, angiogenesis, necessary to support tumor growth, requires blood vessel remodeling, a process that involves apoptosis, which is blocked by IAPs, as has been demonstrated for p35 (Segura et al. FASEB J. 16:833-841, 2002). Therefore, AIAs, such as p35, can be used to enhance anti-tumor activity in cases where a gene encoding a prodrug-activating enzyme or soluble or secretable therapeutic product is delivered to tumor-associated endothelial cells. Moreover, because p35 and certain other caspase inhibitors act as suicide substrates of caspases, inhibiting the caspases in a 1:1 stoichiometric fashion, transient or inducible expression of these apoptosis inhibitors results only in transient inhibition of caspase activity. This presents a particular advantage in the case of neoplastic cells transduced with the caspase inhibitor, insofar as it insures that the caspase pathway of cell death is not disabled permanently, thus further increasing the safety of the methods of the present invention.

Another advantage of the method of the present invention is enhanced immune reaction towards the cells that express the apoptosis inhibitor. Because the cells that express the apoptosis inhibiting agent will not die from apoptosis but will die, for example, from cell death resulting from mitochondrial transition and cytochrome C release, they are likely to undergo a necotic cell death, which can elicit an anti-tumor immune response. Such an anti-tumor immune response is likely to be further augmented by the presence of a foreign antigen, such as the AIAs p35 or p49, resulting in a “systemic bystander effect” that can lead to the destruction of more distant, metastatic cancer cells.

Two major pathways of caspase-dependent apoptosis are known. One pathway is initiated by the formation of a cell death-inducing plasma membrane receptor signaling complex (Scaffidi et al., J Biol. Chem. 274:22532-22538 (1999)), which induces aggregation and activation of the initiator caspase 8 (Ashkenazi and Dixit, Science. 281:1305-1308 (1998); Green, Cell. 94:695-698 (1998); Micheau et al., J Biol. Chem. 274:7987-7992 (1999)). A second apoptotic pathway is triggered by cellular stress such as DNA damage (Green, Cell. 94:695-698 (1998); Sun et al., J Biol. Chem. 274:5053-5060 (1999)) and is primarily associated with a cell death pathway linked to the release of pro-apoptotic molecules from the mitochondria (mitochondrial transition) and the subsequent activation of a distinct initiator caspase, caspase 9 (Green and Reed, Science. 281:1309-1312 (1998); Li et al., Cell. 91:479-489 (1997); Susin et al., J Exp Med. 189:381-394 (1999)). Once activated, caspase 8 and caspase 9 cleave and thereby activate downstream caspase family members, such as caspases 3 and 7 (Slee et al., J. Cell Biol. 144:281-292 (1999)). These downstream effector caspases, in turn, cleave multiple cellular proteins, triggering the phenotypic cellular changes that are associated with apoptosis. Previous studies demonstrate that the mitochondrial-mediated caspase 9 pathway is the key regulatory pathway responsible for CPA-induced tumor cell death (Schwartz and Waxman, Mol Pharmacol. 60:1268-1279. (2001)). There are many other types of pathways of cell death that can be used, but we will exemplify the discussion by focusing on using the above pathways.

We have discovered methods to prolong prodrug activation in a cell that expresses a prodrug activation enzyme (the ‘prodrug-activating factory cell’). For example, the anti-apoptotic factor p35 when expressed in a P450 factory cell prolongs the life span of the P450 factory cell while allowing the factory cell eventually to die.

For example, an apoptosis inhibitor, preferably a caspase inhibitor, such as the Baculovirus protein p35 or p49, is expressed to prolong the life of factory cells that die by a mitochondria-mediated cell death pathway.

p35 and p49 and other inhibitors of apoptosis (AIAs or IAPs) are proteins that bind directly to caspases and inhibit their proteolytic activity, thereby inhibiting apoptosis. IAPs, first discovered in baculovirus, have been identified in both mammals and lower eukaryotes (Deveraux and Reed, Genes Dev. 13:239-252 (1999)). Conserved genes that encode certain IAPs are frequently overexpressed in human cancers and are associated with resistance to therapy. Many IAPs contain a 70-amino acid cysteine- and histidine-rich baculovirus IAP repeat (BIR) and often a zinc-binding RING finger.

In one preferred embodiment of the present invention the AIA is p35. In another preferred embodiment, the AIA is p49. Nucleic acids useful for inhibiting caspase activity are presented in detail, for example, in Zoog et al. (EMBO J. 21: 5130-40 (2002)). The use of p35 is particularly preferred, in part because of its broad-spectrum, pan-caspase inhibitory activity. p35 is also shown herein to have the desirable property of enhancing spread of the infection by a tumor cell-replicating virus, such as the tumor cell replicating adenoviral vector discussed below.

IAPs can inhibit apoptosis induced by a variety of cell death stimuli, including receptor-dependent death signals induced by TNF, Fas and related proteins, chemotherapeutic drugs such as etoposide and taxol, and cellular stress resulting from growth factor withdrawal (Deveraux and Reed, Genes Dev. 13:239-252 (1999)). However, IAPs do not interfere with Bax-mediated release of cytochrome C from mitochondria, suggesting that IAPs block caspase activation and apoptosis downstream of the mitochondrial transition (Roy et al., Embo J. 16:6914-6925 (1997)). Consequently, any of the factors that serve as AIAs can be used to prolong death of the factory cell. The extent to which prodrug activation, or production or secretion of a soluble, therapeutic factor by the factory cell will be increased will vary with the potency of caspase inhibition (Ambrosini et al., Nat. Med., 3:917-21 (1997)).

IAPs have been cloned and characterized in a broad range of organisms, including viral, fly, nematode, yeast and mammalian species. Particularly useful AIAs according to the present invention are p35 or p49, both Baculovirus proteins. Examples of mammalian AIAs useful according to the present invention include: XIAP, hIAP1 (Uren et al., Proc. Nat. Acad. Sci. 93: 4974-4978, 1996), hIAP2 (Rothe et al., Cell 83: 1243-1252, 1995), Naip (Roy, et al., Cell 80: 167-178, 1995), Bruce, Survivin (Ambrosini et al., Nature Med. 3: 917-921, 1997), and pIAP (reviewed by Deveraux and Reed, Genes Dev., 13(3):239-52 (1999)). Other IAPs useful according to the present invention include CrmA, CiIAP, OpIAP/CpIAP/AcIAP, ASFIAP, DIAP1, DIAP2, CeIAP1, CeIAP2, SpIAP and ScIAP (Deveraux and Reed, Genes Dev., 13:239-52 (1999)), as well as Bcl-2 family proteins with anti-apoptotic properties, including Bcl-2, Bcl-X_{L} and Mcl (Strasser et al., Biochim Biophys Acta 1333: F151-F178 (1997).

Moreover, different caspase inhibitors can be selected in different systems. For example, TRAIL-induced apoptosis may be more effectively inhibited by CrmA or XIAP than by p35. On the other hand, etoposide-induced apoptosis may be more effectively inhibited by XIAP (Ryan et al., Biochem J 366:595-601 (2002)).

Although the inhibition of apoptosis does not prevent the cell from eventually dying, it may be useful as an added safety measure to further ensure that the IAP-expressing factory cell will eventually die. Therefore, the IAP may be expressed from a regulatable promoter, with the regulating agent maintained only as long as is desired to regulate expression of the IAP so as to prolong the life of the factory cell. Alternatively, expression of the AIA from a non-regulatable promoter can be followed by a regulatable expression of a factor such as Smac/Diablo or an anti-sense or siRNA to the AIA. Smac/Diablo can inactivate IAPs, and can thereby relieve the inhibition of cell death that is conferred by the AIA (Du et al., Cell 102: 3342 (2000); Verhagen et al., Cell 102: 43-53 (2000)), while expression of IAPs can be inhibited at the mRNA level using anti-sense nucleic acid or siRNA targeting the IAP. Alternatively, factory cells that are of type I can be treated with a death receptor ligand to insure their ultimate death. Numerous other scenarios are possible.

Regulated expression of the AIA can be achieved by using an regulatable promoter to control the level of expression of the AIA. A promoter is a DNA sequence that directs the transcription of a gene. Typically, a promoter is located in the 5′ flanking region of a gene, proximal to the transcriptional start site. If a regulatable promoter is used, then the rate of transcription of the gene, and hence the level of gene expression, can be either increased or decreased in response to the regulating agent. Regulatable expression means one can increase or decrease expression dependency on what one wants.

According to the present invention, it may be desirable to increase AIA gene expression initially, to prolong cell death and thereby provide for the desired increase in production of activated drug metabolites, or of secreted, or soluble therapeutic factor. Subsequently, it may be desirable to decrease expression of the AIA in order to ensure the death of the AIA-expressing tumor cell. The possibility of long-term survival of a tumor cell population transduced with p35 can be greatly diminished by placing the p35 gene under the control of a strong, regulatable promoter, for example, one that can be repressed. One such example is the promoter utilized in the ‘Tet-off’ expression system (Clontech, Inc., Palo Alto, Calif.). As explained in more detail in the following examples, the Tet-off system is well suited for achieving a high degree of control of transgene expression, which can be suppressed in a wide range of cells both in vitro and in vivo. Tetracycline is a well-tolerated antibiotic, and near complete suppression of gene expression can be achieved at doses that are very low and have no cytotoxicity or other significant effects on cell proliferation or animal growth, even with continuous treatment. For example, adenoviral vectors that incorporate a regulated transgene in the viral E1 deletion region and the tetracycline-responsive transcriptional activator gene in the E3 deletion region and can be used to deliver the p35 cDNA (1 kb in length) in combination with a minimal CMV-P450 expression cassette (˜2.6 kb) or a CMV-P450-IRES-P450R expression cassette (˜5.3 kb). Use of such a vector provides for tetracycline suppression of p35 expression, thereby enabling the P450 prodrug- and P450-dependent killing of any residual tumor cells that may have initially been protected by p35 from cytotoxicity of the P450-activated prodrug. This Tet-off adenovirus provides for very tight regulation, with 20- to 500-fold suppression of transgene expression achievable by treatment with very low tetracycline doses. Any such low level, leaky expression of p35 would not reach the threshold required to block prodrug-induced cell death. (Mizuguchi H & Hayakawa T. (2002) J Gene Med 4: 240-247). In contrast, the rate of transcription is not regulated or is largely unregulated by an inducing agent if the promoter is a constitutive promoter. Examples of other regulatable promoters are described in Haviv et al., Adv Drug Deliv Rev 53:135-54 (2001).

Alternatively, an inhibitor of death receptor-mediated cell death is expressed to prolong the life of factory cells that would otherwise die by a death receptor-mediated cell death pathway. The factory cells may be producing either an activated prodrug or a soluble, or secretable therapeutic factor that kills the cells by a death receptor-mediated cell death pathway. Examples of such death receptor inhibitory factors are FLIPs, decoy receptors and dominant-negative FADD (Fas-associated death domain protein).

FLIPs (Fas-associated death domain-like ice inhibitory proteins) are proteins that are structurally similar to caspase-8 and interfere with the recruitment and activation of caspase-8 to the death receptors. FLIPs slow down but do not block the ultimate death receptor response (Baker and Reddy (1998)). FLIPs are encoded by viruses (v-FLIPs) (Zorning et al, Biochim Biophys Acta 1551: F1-37 (2001)) and by mammalian cells (c-FLIPs). Eleven cFLIP isoforms have been described, all of which can inhibit the Fas-mediated cell death pathway (Djerbi et al, Scand. J. Immunol. 54: 180-189 (2001).

Decoy receptors, including those designated DcR1, DcR2, DcR3 and osteoprotegrin, can also slow transmission of the death signal without completely blocking it (Ashkenazi and Dixit (1999)). Similarly, dominant-negative FADD can be used to inhibit death receptor-dependent apoptotic responses induced by the prodrug ganciclovir when activated by herpes simplex virus thymidine kinase (Beltinger et al, PNAS 96: 8699-8704 (1999)).

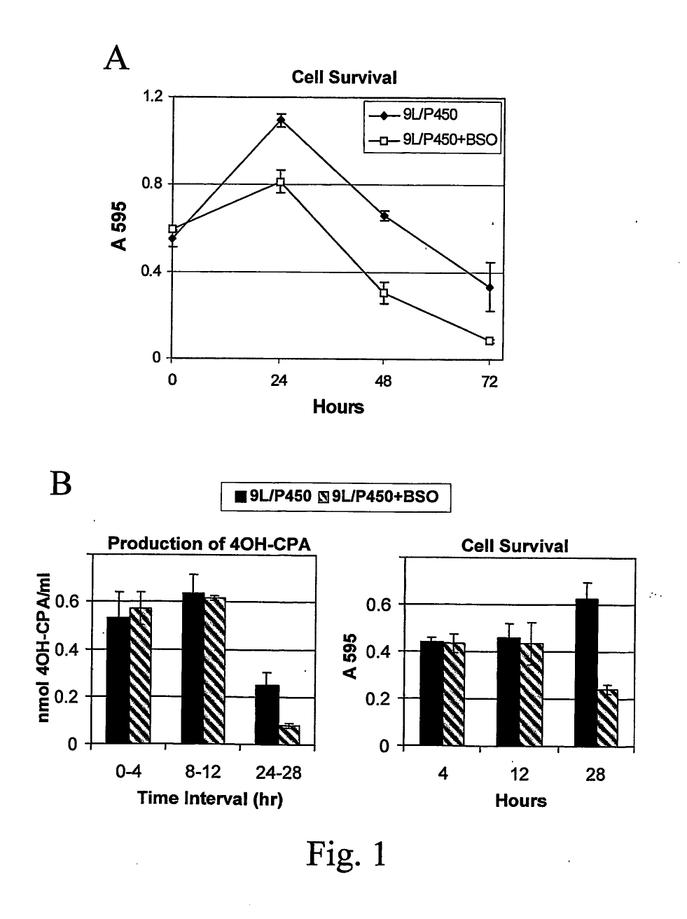

The AIA or IAP may be delivered into a cell using any method known to one skilled in the art that will accomplish the goal including genetic engineering and direct delivery of proteins or nucleic acids without genetic engineering. For example, if the AIA is a protein, for example an antibody or a soluble receptor decoy, a method such as described in Kreis et al., EMBO J. 5: 931-941, (1986)), may be used. Kreis describes a method by which a protein can be injected intracellularly using microinjection. Another method of delivering a protein intracellularly uses endocytosis to have the protein enter the cell, for example, chemical conjugates containing an antibody conjugated to the protein (i.e., an anti-apoptotic) desired to be delivered into the cell. The antibody is intended to bind extracellularly to the cell, and the process of endocytosis results in bringing the desired protein into the cell. See particularly FIG. 1 of Oeltmann and Frankel, FASEB J 5:2334-2337 (1991)) and Seetharam et al., J Biol Chem 266: 17376-17381 (1991)).

RNAi has been shown to be a powerful tool for manipulating gene expression in cells (Hannon, Nature 418:244-251 (2002)). The technology arose from the observation that exogenous double-stranded RNAs induce gene silencing in plants and Caenorhabditis elegans. These double-stranded RNAs are processed into small interfering RNAs (siRNAs), which are incorporated into a conserved cellular machinery that mediates the suppression of homologous genes. Recently, small non-coding RNAs have been identified that can act as endogenous regulators of gene expression. These micro RNAs typically form stem-loop structures, essentially short double-stranded RNAs, which enter the RNAi pathway (Knight et al., Science 293: 2269-2271 (2001); Ketting et al., Genes Dev, 15:2654-2659 (2001); Hutvagner et al., Science 293 834-838 (2001); Grishok et al., Cell 106: 23-34 (2001)). siRNAs, modeled after micro RNAs, can be expressed from viral vectors to induce stable suppression of gene expression in cultured mammalian cells (Paddison and Hannon, Cancer Cell 2:17-23 (2002)) and in various tissues in vivo (Add Refs: McCaffrey et al., Nature 418: 38-39 (2002); Lewis et al Nat Genetics 32: 107-108 (2002)). Methods for preparing and delivering sequence specific interference RNAs into cells are presented, for example, in the U.S. Patent Application Publication No. 20020162126, which is herein incorporated by reference in its entirety.

Therefore, in one embodiment of the present invention, an siRNA targeting an apoptosis inducing gene, for example a member of a caspase family of proteins or other pro-apoptotic factor is introduced into the cell together, before or after introducing a prodrug activating enzyme encoding gene. The sequence specific dsRNA construct constituting the siRNA may be directly introduced into the cell (i.e., intracellularly); or introduced extracellularly into a cavity, interstitial space, into the circulation of an organism or introduced orally. Methods for oral introduction include direct mixing of RNA with food of the organism, as well as engineered approaches in which a species that is used as food is engineered to express an RNA, then fed to the organism. Physical methods of introducing nucleic acids include injection directly into the cell, gene or extracellular injection into the organism of an RNA solution. The double-stranded structure may be formed by a single self-complementary RNA strand or two complementary RNA strands. RNA duplex formation may be initiated either inside or outside the cell. siRNAs can also be delivered using viral vectors, including, but not limited to, retroviral and adenoviral vectors, which may either be replication defective, conditionally replicating or replication sufficient.

Further, because caspases are a family of proteins with homologous domains, one siRNA can be designed that targets several different caspase mRNAs at the same time. For example, in one preferred embodiment, the siRNA is directed to target mRNA encoding amino acids C(L/V)I(I/V)NN (SEQ ID NO: 5), L(S/T)HG (SEQ ID NO: 6), or KPKLFFIQAC (SEQ ID NO: 7), which are homologous at least between caspase 8 (nucleic acid accession number gi:12862693), caspase 9 (gi:27802691) and caspase 10 (gi:12862685). Other homologous regions in different apoptosis inhibiting can be easily identified using the PAIRWISE BLAST algorithm at http://www.ncbi.nlm.nih.gov/blast/bl2seq/bl2.html using default settings.

Antisense nucleic acids and oligonucleotides targeted against apoptosis inducing molecules, such as caspase 8 and caspase 9, useful according to the invention can be constructed using chemical synthesis and enzymatic ligation reactions using procedures known in the art. The antisense nucleic acid or oligonucleotide can be chemically synthesized using naturally occurring nucleotides or variously modified nucleotides designed to increase the biological stability of the molecules or to increase the physical stability of the duplex formed between the antisense and sense nucleic acids e.g. phosphorothioate derivatives and acridine substituted nucleotides can be used. Alternatively, the antisense nucleic acids and oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1 (1) 1986.

A catalytic RNA targeting a specific apoptosis inducing agent, such as caspase targeting, ribonuclease activity, can be selected from a pool of RNA molecules. See for example Bartel and Szostak, Science 261: 1411-1418 (1993). Such RNA molecules may also serve as IAPs useful according to the present invention.

Aptamers targeting apoptosis inducing proteins, for example caspases, can be produced using the methodology disclosed in a U.S. Pat. No. 5,270,163 and WO 91/19813.

All the above-discussed approaches allow a prolonged expression of the therapeutic gene, and hence prolonged production of the therapeutic agent by the factory cell. This is accomplished by inhibition of mitochondria-dependent cell death, in the case of a prodrug such as CPA, which activates the mitochondrial cell death pathway; or by inhibition of death receptor-dependent cell death, in the case of a death receptor ligand, such as TNFα, TRAIL or Fas ligand, or prodrugs such as ganciclovir or 5-fluorocytosine, which activate receptor-dependent cell death pathways. The pathways of apoptosis are well known for many anti-cancer agents, including anti-cancer prodrugs. The nature of the apoptotic pathway (e.g., mitochondrial pathway vs. cell surface death receptor pathway) can readily be established for known and for novel anti-cancer agents and prodrugs using experimental methods presently established and well known to those skilled in the art.

To implement this strategy using both a nucleic acid encoding a prodrug-activating enzyme (suicide gene), or a soluble, or secretable therapeutic factor, and a nucleic acid encoding an anti-apoptotic agent in a safe and effective manner, regulation of the expression of the prodrug activation gene, or the soluble or secretable factor gene, and the anti-apoptotic gene should be tightly linked, e.g., by use of an IRES (internal ribosome entry site) sequence, thereby precluding the possibility that the anti-apoptotic factor would be expressed in tumor cells in the absence of the prodrug activating enzyme or therapeutic factor.

Alternatively, the prodrug-activating enzyme gene, or the therapeutic factor gene, and the anti-apoptotic factor can be expressed using cellular vectors, including microencapsulated cells. The use of microencapsulated cells circumvents the possibility that the anti-apoptotic factor might inadvertently confer drug resistance to the tumor cell, insofar as no direct gene therapeutic intervention in the target tumor cell is involved. Alternative suicide or therapeutic factor gene/anti-apoptotic approaches could also be implemented using microencapsulated cells. For example, local delivery of a death receptor ligand, such as Trail, could be achieved using microencapsulated cells that express recombinant Trail together with high levels of the Trail decoy receptor. In this way the encapsulated cells would secrete Trail into the tumor milieu without themselves succumbing to Trail-induced apoptosis.

Death of the factory cell can subsequently be accomplished using any of a number of methods. One method involves the inducible expression of a factor that activates an alternative death pathway. For example, the anti-apoptotic factor Bcl-2 can be used to prolong the life of a P450-expressing factory cell, followed by the inducible expression of a death receptor ligand, such as Trail. Alternatively, a Trail decoy receptor can be used to protect a Trail-expressing cell from Trail-induced suicide, followed by inducible expression of a prodrug activating enzyme and prodrug treatment. Other Bcl-2 family members with anti-apoptotic activity may also be used. These include, but are not limited to, Bcl-XL, Bcl-w, and Mcl (Strasser et al., Biochim Biophys Acta 1333: F151-F178 (1997)). Similar effects can be achieved using anti-sense or siRNA targeted to pro-apoptotic factors that contribute to the apoptotic response. These include, but are not limited to, Bcl-2 family members Bax, Bak, Bcl-Xs, Bad, Bik and Bid (Strasser et al., (1997)).

In the case of prodrug activation therapy directed at cancer treatment, the prodrug activation gene should be expressed at a higher level, and preferably exclusively, in the target tumor tissue than in drug-sensitive host tissues. This expression can be directed to the tumor's neoplastic cells or to non-neoplastic, tumor-associated cells, such as tumor-associated endothelial cells. Moreover, the gene should be expressed at a level that is sufficient to generate a therapeutic level of activated prodrug e.g., a level that is toxic to tumor cells or to tumor-associated endothelial cells. Additionally, for maximal discrimination between host tissues and tumor cells transduced by the suicide gene, the prodrug should be intrinsically inactive, or should at least be substantially less cytotoxic than the activated drug. Finally, the prodrug must be a substrate for the activating enzyme under physiological conditions (Springer and Niculescu-Duvaz, J Clin Invest. 105:1161-1167 (2000)). Because expression of the prodrug activation enzyme in vivo cannot readily be achieved in 100% of the cells of a target tumor using current gene delivery technologies, the enzyme-prodrug combination preferably takes advantage of a strong bystander effect, whereby the active agent (activated prodrug or soluble death receptor ligand) diffuses from the tumor cell or tumor-associated cell in which it is formed into neighboring tumor cells or tumor-associated endothelial cells that do not express, for example, the prodrug-activation enzyme, in quantities that are sufficient to induce a bystander cytotoxic response. Therefore, in cases where the gene is to be expressed intracellularly, as opposed to on the surface of the tumor cell, the prodrug and its active metabolite must both be freely diffusible across cell membranes.

Soluble, or secretable therapeutic agents useful according to the present invention include agents with anti-angiogenic, cytotoxic or immune modulatory activity. Examples include, but are not limited to the following: anti-angiogenic factors, such as endostatin, angiostatin, VEGF antibody, VEGF receptor-derived ectodomain, tumstatin and other integrin-binding or integrin-inhibiting molecules, 16 kd prolactin fragment, platelet factor 4 and antibody, anti-sense agents or siRNA directed against angiogenic factors and other anti-angiogenic agents cited by Kerbel and Folkman in Nature Reviews 2: 727-739 (2002); members of the tumor necrosis factor superfamily, including TNFα, Fas ligand and Trail (Zhou et al, (2002) Immunol Res 26:323-336); and immune modulatory cytokines, including interferon α, interferon β, and interleukins 2, 12 and 18 (Wigginton and Wiltrout, (2002) Expert Opin Biol Ther 2:513-524). The target cell is one that is located near where you want the expression. For example, when the target is a tumor the target cell may be a neoplastic cell or a non-neoplastic cell within the tumor, such as a tumor-associated endothelial cell. The key is that the cell expressing the therapeutic agent is located in the vicinity of neoplastic or tumor-associated endothelial cells, whose killing is known to be associated with an anti-tumor response.

Examples of enzyme prodrug systems useful according to the present invention include, but are not limited to the following (Rigg and Sikora (1997); Niculescu-Duvaz and Springer; Aghi et al, (2000)): cytochrome P450 in combination with cyclophosphamide (CPA), ifosfamide and other P450 prodrugs, as well as bioreductive drugs that are activated by P450 and/or by NADPH-P450 reductase; thymidine kinase/ganciclovir, acyclovir and related prodrugs; cytosine deaminase/5-fluorocytosine; nitroreductase/CB 1954 and other aromatic nitro prodrugs; thymidine phosphorylase/5′-deoxy-5-fluorouridine; purine nucleoside phosphorylase/6-methylpurine-2′-deoxynucleoside; alkaline phosphatase/etoposide phosphate; carboxypeptidase A/methotrexate-(phenyl)alanine; carboxypeptidase G2/benzoic acid mustard-glucuronide; linamarase/amygdalin; beta-lactamase/cephalosporin-mustard carbamate; xanthine oxidase/xanthine; guanine phosphoribosyl transferase (GPT)/6-thioxanthine; deoxycytidine kinase/cytosine arabinoside; uracil phosphoribosyltransferase/5-fluorouracil; carboxylesterase/irinotecan (CPT-11); folylpolyglutamate synthetase/edatrexate.

Preferably, a prodrug activation gene therapy strategy is based upon a cytochrome P450 gene (“CYP” or “P450”) in combination with a cancer chemotherapeutic agent that is activated through a P450-catalyzed monooxygenase reaction (Chen and Waxman, Cancer Research 55:581-589 (1995); Wei et al., Hum. Gene Ther. 5:969-978 (1994); U.S. Pat. No. 5,688,773). A further increase in prodrug activation, and a broader spectrum of prodrug substrate activity may be achieved by combining P450 gene transfer with NADPH-P450 reductase gene transfer. (U.S. Pat. No. 6,207,648). An advantage of the P450-based drug activation strategy is that it utilizes a mammalian prodrug activation gene rather than a bacterially or virally derived gene. In addition, the P450 system utilizes chemotherapeutic drugs that are established and widely used in cancer therapy. Investigational anti-cancer prodrugs, and novel prodrugs designed to be activated by way of P450 metabolism can also be used in this therapy.

The methods of killing neoplastic cells using cytochrome P450-based gene therapy and the transfer of the cytochrome P450 gene product to a neoplastic cell, alone or in combination with NADPH-cytochrome P450 reductase, are described in detail in U.S. Pat. No. 6,207,648 and U.S. Pat. No. 5,688,773, which are herein incorporated by reference in their entirety.

Many anti-cancer drugs are prodrugs that undergo P450-catalyzed metabolism, resulting in the formation of metabolites that have increased activity against tumor cells. P450 prodrugs include several commonly used cancer chemotherapeutic drugs and other therapeutic agents, such as CPA, ifosfamide, dacarbazine, procarbazine, thio-TEPA, etoposide, 2-aminoanthracene, 4-ipomeanol, tamoxifen, acetaminophen (LeBlanc and Waxman, Drug Metab. Rev. 20:395-439 (1989); Ng and Waxman, Intl. J. Oncology 2:731-738 (1993); Goeptar et al., Cancer Res. 54:2411-2418 (1994); van Maanen, et al., Cancer Res. 47:4658-4662 (1987); Dehal et al., Cancer Res. 57:3402-3406 (1997); Rainov et al., Human Gene Therapy 9:1261-1273 (1998)), cyclic phosph(on)ate ester-containing P450 prodrugs (U.S. Pat. No. 6,312,662), methoxymorpholinyl doxorubicin (Quintieri et al, Cancer Res 60:3232-3238) and tegafur (Komatsu et al., Drug Metab Dispos 28:1457-1463 (2000)). Bioreductive anti-cancer prodrug activation is also catalyzed by cytochrome P450 enzymes. Examples of such anti-cancer drugs include Adriamycin, mitomycin C, tetramethylbenzoquinone (Goeptar et al., Crit. Rev. Toxicol. 25:25-65 (1995); Goeptar et al., Mol. Pharmacol. 44:1267-1277 (1993)), AQ4N and tirapazamine (Patterson et al., Anti-Cancer Drug Design, 14:473-486 (1999); Jounaidi and Waxman, Cancer Res 60:3761-3769 (2000)).

The anti-apoptotic agent (AIA or IAP) can be introduced to the cells expressing a therapeutic gene, for example a prodrug activating enzyme, using any means known to one skilled in the art. The IAP may be in the form of an isolated and purified protein or a recombinant and purified protein, an antibody against IAP or an antigenic fragment thereof, a small molecule, a single- or double-stranded deoxynucleic acid (RNA or DNA) either encoding the IAP, the antibody against the IAP, or antisense nucleic acid or siRNA targeted to an IAP.

Antibodies against the IAP's useful according to the present invention can be intact monoclonal or polyclonal antibodies, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. See e.g. U.S. Pat. No. 6,235,883.

Examples of viral vector systems utilized in the gene therapy art include the following: retroviruses (Vile, R. G., supra; U.S. Pat. Nos. 5,741,486 and 5,763,242); adenoviruses (Brody, S. L., et al., Ann. N.Y. Acad. Sci. 716: 90-101 (1994); Heise, C. et al., Nat. Med. 3:639-645 (1997)); adenoviral/retroviral chimeras (Bilbao et al., FASEB J 11:624-634 (1997); Feng et al., Nat. Biotechnol. 15:866-870 (1997)); adeno-associated viruses (Flotte and Carter, Gene Ther. 2:357-362 (1995); U.S. Pat. No. 5,756,283); herpes simplex virus I or II (Latchman, Mol. Biotechnol. 2:179-195 (1994); U.S. Pat. No. 5,763,217; Chase et al., Nature Biotechnol. 16:444-448 (1998)); parvovirus (Shaughnessy et al., Semin Oncol. 23:159-171 (1996)) reticuloendotheliosis virus (Donburg, Gene Therap. 2:301-310 (1995)). Also of interest in the art, is the development of extrachromosomal replicating vectors for gene therapy (Calos, Trends Genet. 12:463-466 (1996)). Other viruses that can be used as vectors for gene delivery include poliovirus, papillomavirus, pox viruses such as orthopoxes including vaccinia virus and avipox including fowl pox and canary pox, lentivirus, as well as hybrid or chimeric vectors incorporating favorable aspects of two or more viruses (Nakanishi, Crit. Rev. Therapeu. Drug Carrier Systems 12:263-310 (1995); Zhang et al., Cancer Metastasis Rev. 15:385401 (1996); Jacoby, et al., Gene Therapy 4:1281-1283 (1997)). Retroviruses and adenoviruses are the preferred viral vectors for gene delivery. Other suitable viral vectors will be readily apparent to the skilled artisan.

The vector will include one or more promoters or enhancers, the selection of which will be known to those skilled in the art. Suitable promoters include, but are not limited to, the retroviral long terminal repeat (LTR), the SV40 promoter, the human cytomegalovirus (CMV) promoter, and other viral and eukaryotic cellular promoters known to the skilled artisan. Examples of DNA enhancer sequences include the tumor tissue-specific enhancers, described below.

Guidance in the construction of gene therapy vectors and the introduction thereof into affected animals for therapeutic purposes may be obtained in the above-referenced publications, as well as U.S. Pat. Nos. 5,631,236, 5,688,773, 5,691,177, 5,670,488, 5,529,774, 5,601,818, and WO 95/06486.

An example of a vector useful according to the present invention is an adenovirus vector. Unlike plasmids and other viral vectors (e.g., herpes simplex virus), adenoviral vectors achieve gene transfer in both dividing and nondividing cells, with high levels of protein expression in cardiovascular relevant sites such as myocardium, vascular endothelium, and skeletal muscle. Furthermore, the gene transferred by an adenoviral vector functions in an epichromosomal position and thus carries little risk of inappropriately inserting the transferred gene into a critical site of the host genome. The adenoviral vector also desirably is deficient in at least one gene function required for viral replication. Preferably, the adenoviral vector is deficient in at least one essential gene function of the E1, E2, and/or E4 regions of the adenoviral genome. More preferably, the vector additionally is deficient in at least part of the E3 region of the adenoviral genome (e.g., an XbaI deletion of the E3 region). Recombinant adenovirus can be delivered to cultured cells by simply adding the virus to the culture media. Infection of host animals/humans can be achieved by directly injecting the viral particles into the bloodstream or into the desired tissue. The half-life of the virus in serum can be extended by complexing the virus with liposomes (e.g. Lipofectin, Life Technologies) or polyethylene glycol. The adenovirus vector normally enters the cell through an interaction between the knob domain of the viral fiber protein and the coxsackievirus and adenovirus receptor, CAR. The viral vector can be directed to specific cells, or to cells which do not express the CAR, by genetically engineering the virus to express a ligand specific to a certain cell receptor or by retargeting the virus by specific mutation, e.g., of the viral fiber protein, to decrease its dependence on CAR for cellular entry (Mizuguchi and Hayakawa, Canc Gene Therap 9: 236-242 (2002); Vigne et al., Gene Therapy 10: 153-162 (2003).

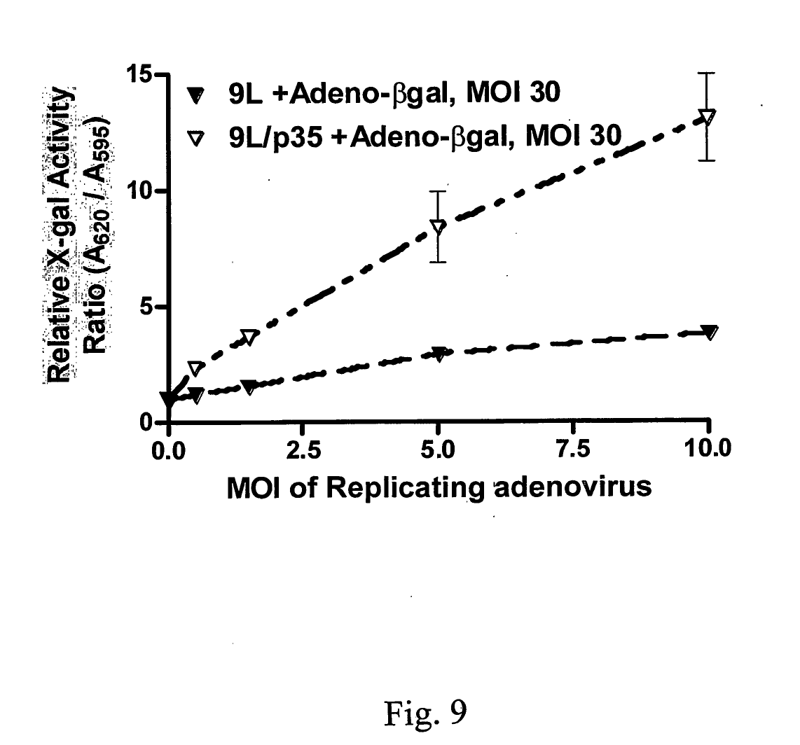

In one preferred embodiment, the viral vector is a tumor-cell replicating adenoviral vector such as an E1b region, 55 kd protein-deleted adenovirus. Conditional replicating adenoviruses, such as the E1b-55 kd deleted adenovirus Onyx-015, are oncolytic, and can induce tumor cell death by both apoptotic and necrotic mechanisms (Bischoff et al., Science 274: 373-376 (1996)). The Onyx virus replicates in a tumor-selective manner and has shown promise in late-stage clinical trials for treating a variety of malignancies when given in combination with chemotherapy (Ries & Kom, Br J Cancer 86: 5-11 (2002)). In normal host cells infected with wild-type adenovirus, viral E1a proteins induce rapid death by an apoptotic mechanism that requires the pro-apoptotic factor Bax (Chinnadurai, Semin Virol 8: 399-408 (1998); Lomonosova et al., J Virol 76: 11283-11290 (2002)). Two E1b-region viral proteins cooperate to suppress this apoptotic response, thereby facilitating viral replication. E1b-55k binds to and inactivates cellular p53, while E1b-19k acts as a functional homolog of the cellular anti-apoptotic factor Bcl-2 (Burgert et al., Curr Top Microbiol Immunol 269: 273-318 (2002); Cuconati & White, Genes Dev 16: 2465-2478 (2002)). In normal host cells infected with the E1b-55k-deleted Onyx-015 virus, however, the host cell-protective functions of p53 are preserved, and virus-induced cell cycle arrest and apoptosis occur, severely limiting host cell viral replication and viral spread (Ganly et al., Gene Ther 8: 369-375 (2001)). By contrast, when tumor cells are infected with Onyx-015, cellular defects in p53, or in p53 pathway components such as p14/ARF and Mdm2, enable the virus to replicate efficiently and induce cell death by a slow, cytolytic process (Ries & Korn, Br J Cancer 86: 5-11 (2002); McCormick, Oncogene 19: 6670-6672 (2000)). This cell death is synergistically enhanced by chemotherapeutic drugs, as has been demonstrated in preclinical studies and in phase II clinical trials (Heise et al., Nat Med 3: 639-645 (1997); You et al., Cancer Res 60: 1009-1013 (2000); Khuri et al., Nat Med 6: 879-885 (2000)). The therapeutic impact of a replicating virus, including tumor cell-replicating adenovirus and herpes virus, may therefore be enhanced by arming the virus with a prodrug-activating enzyme, such as cytochrome P450 (Pawlik et al., Canc Res 60: 2790-2795(2000)). Other studies have shown that tumor cell-replicating viruses can be used as helper viruses to enhance tumor cell spread and replication, both in vitro and in vivo, of adenoviruses that are otherwise replication-defective (Habib et al., Cancer Gene Ther 9: 651-654 (2002); Motoi et al., Hum Gene Ther 11: 223-235 (2000)). Despite the fact that adenovirus already has an anti-apoptotic viral E1b-19k protein that plays in increasing viral replication and spread (Cuconati & White, Genes Dev 16:2465-2478 (2002)), further increases in tumor cell spread may be achieved by incorporating a downstream-acting anti-apoptotic factor, such as the pan-caspase inhibitor p35 (Clem, Cell Death Differ 8: 137-143 (2001)), as shown herein (FIG. 9, below).

Mouse studies involving replication-defective and replication-conditional adenovirus: Replication-defective adenovirus type 5, alone or in combination with a tumor-replicating adenovirus (e.g., E1b-55k-deleted adenovirus), will be used to transfer P450 and p35 transgenes to solid tumors (˜100-150 mm³) growing s.c. in scid mice. Animal experimentation procedures will be performed in accordance with good biohazard/RDNA practice, and consistent with the Biohazard Level 2 (BL-2N) approval obtained for these studies. In a typical experiment, adenovirus (up to 10⁹ pfu/injection) will be administered intratumorally or systemically, by tail vein injection in a vol of 50 μl, daily×5. CPA treatment will use standard drug treatment protocols. Human clinical trial using the Onyx adenovirus have established that this virus can be administered safety to humans at exceptionally high doses (up to 2×10¹³ pfu/patient) without significant toxicity (Nemunaitis et al., Gene Ther 8: 746-759 (2001)).

Another preferred vector useful according to the present invention is a poxvirus vector. See, e.g., U.S. Pat. No. 5,656,465. Compared to other systems such as retrovirus vectors (including lentiviral vectors), adenoviral vectors, and adeno-associated virus vectors, the large genome of poxviruses enables large genes to be inserted into pox-based vectors. Particularly useful poxvirus vectors include the more attenuated orthopox such as NYVAC (U.S. Pat. No. 5,364,773) and modified vaccinia Ankara (MVA) and avipox such as fowl pox and canary pox. Other pox include suipox. MVA is an attenuated, replication-defective virus, which is restricted to replication primarily in avian cells. Comparison of the MVA genome to its parent, CVA, revealed 6 major deletions of genomic DNA (deletion I, II, III, IV, V, and VI), totaling 31,000 basepairs. (Meyer et al., J. Gen. Virol. 72: 1031-8 (1991)). MVA has been administered to numerous animal species, including monkeys, mice, swine, sheep, cattle, horses and elephants with no local or systemic adverse effects. Over 120,000 humans have been safely vaccinated with MVA by intradermal, subcutaneous or intramuscular injections. MVA has also been reported to be avirulent among normal and immunosuppressed animals (Mayr et al., Zentralb. Bakteriol. 167:375-90 (1978). For example, MVA contains 6 natural deletion sites, which have been demonstrated to serve as insertion sites for therapeutic genes and which are accordingly useful in the present invention. See e.g. U.S. Pat. No. 5,185,146, and U.S. Pat. No. 6,440,422. Accordingly, these more attenuated strains are particularly attractive poxviruses for use as vectors for gene therapy to deliver the apoptosis inhibiting and therapeutic genes into cells. Yet another preferred vector is AAV. There are a number of strains. Preferably, one uses an attenuated AAV. Still other methods of delivering include gene gun, liposomes and naked DNA which are discussed in more detail later.

Generally, methods are known in the art for viral infection of the cells of interest. The virus can be injected into a patient bearing a neoplasm, either at, into, or near the site of neoplastic growth. Preferentially, the treatment will be by direct intraneoplastic inoculation. For tumors in the brain, magnetic resonance imaging (MRI), computerized tomography (CT), or other imaging guided stereotactic technique may be used to direct inoculation of the vector. The tumor may also be resected prior to treatment with the vectors of the invention.

The pharmaceutical compositions of the present invention would be advantageously administered in the form of injectable compositions. A typical composition for such purpose would comprise a pharmaceutically acceptable vehicle. Other pharmaceutically acceptable carriers include aqueous solutions, non-toxic excipients, including salts, preservatives, buffers and the like. See, Remington's Pharmaceutical Sciences (18th ed.), Mack Publishing Co. (1990). Examples of non-aqueous solvents are propylene glycol, polyethylene glycol, vegetable oil and injectable organic esters such as ethyloleate. Aqueous carriers include water, aqueous solutions, saline solutions, parenteral vehicles such as sodium chloride, Ringer's dextrose, etc. Intravenous vehicles include fluid and nutrient replenishers. The pH and exact concentration of the various components of the pharmaceutical composition are adjusted according to routine skills in the art (Goodman and Gilman, The Pharmacological Basis for Therapeutics (8th edn.) Pergamon Press (1990)).

Typically, the vector would be prepared as an injectable, either as liquid solutions or suspensions; solid forms suitable for solution in, or suspension in, liquid prior to injection may also be prepared. The preparation also may be emulsified. The active immunogenic ingredient is often mixed with an excipient which is pharmaceutically-acceptable and compatible with the active ingredient. Suitable excipients are, for example, water, saline, dextrose, glycerol, ethanol, or the like and combinations thereof. In addition, if desired, the preparation may contain minor amounts of auxiliary substances such as wetting or emulsifying agents, pH-buffering agents, adjuvants or immunopotentiators.

In general, the virus is provided in a therapeutically effective amount to infect and target cells so that they will produce the therapeutic gene, for example a prodrug activating enzyme, and the apoptosis inhibiting agent. The quantity of the vector to be administered, both according to number of treatments and amount, will also depend on factors such as the clinical status, age, and weight of the subject to be treated, the capacity of the subject's immune system to synthesize antibodies, and available volume. Precise amounts of active ingredient required to be administered depend on the judgment of the gene therapist and will be particular to each individual patient. Generally, the viral vector is administered in titers ranging from about 1×10⁵ to about 1×10¹⁰ colony forming units (cfu) per ml, although ranges may vary. Preferred titers will range from about 1×10⁶ to about 1×10⁹ cfu/ml.

In one embodiment, a packaging cell line is transduced with a retroviral vector carrying the therapeutic gene (prodrug activation enzyme or soluble, or secretable therapeutic factor) and/or the IAP cDNA or gene to form a producer cell line. The packaging cells may be transduced by any means known in the art, including, e.g., electroporation, CaPO₄ precipitation, or the use of liposomes. Examples of packaging cells that may be transfected include, but are not limited to, BOSC23, Bing, PE501, PA317, .PSI.-2, .PSI.-AM, PA12, T19-14X, VT-19-17-H2, .PSI.-CRE, .PSI.-CRIP, GP+E86, GP+envAm12, and DAN cell lines. Guidance on retroviral producing packaging cells and how to construct them can be found in Short et al., J. Neurosci. Res. 27:427-433 (1990); Miller, Human Gene Ther. 1:5-14 (1990); Danos “Construction of Retroviral Packaging Cell Lines,” in Methods in Molecular Biology (M. Collins, ed), Vol. 8, The Humana Press Inc., Clifton, N.J., 17-26 (1991); Murdoch, et al., Gene Therapy 4:744-749 (1997); and U.S. Pat. Nos. 5,529,774, and 5,591,624.

Retroviral vectors can also be successfully packaged with a vesicular stomatitis virus (VSV) envelope glycoprotein G (“pseudotyping”). These vectors are more stable and can be concentrated to 10⁹ cfu/ml, allowing them to be injected directly (Burns et al., Proc. Natl. Acad. Sci. USA 90:8033-8037 (1993)).

Viral producer cells can be grafted near or into the tumor in an amount effective to inhibit growth of, or kill, the neoplastic cells. Direct injection of high titer retroviral producer cells (Murdoch et al., Gene Ther. 4:744-749 (1997); Onodera et al., Hum Gene Ther. 8:1189-1194 (1997)) can allow for efficient in situ infection with the retroviral sequences (Rainov et al., Cancer Gene Ther. 3:99-106 (1996); Ram et al., Cancer Res. 53:83-88 (1993)). Producer cells injected intratumorally do not generally migrate from the site of injection. Moreover, although they may be rejected by the host, this does not occur for 5-10 days, by which time retroviral infection of nearby tumor cells will have occurred (Ram et al., J Neurosurg. 79:400-407 (1993)). In general, vector producer cell (VPC) dosages range from about 2.5×10⁸ VPCs to about 1×10⁹ VPCs. The exact amount of producer cells will ultimately be determined by the skilled artisan based on numerous factors, including, but not limited to, the available injectable volume, clinical status of the patient, and tumor type and size.

Preferably, the viral genomes of the viral vectors used in the invention should be modified to remove or limit their ability to replicate, however, replication conditional viruses will also be useful in the present invention, as will replicating vectors that are capable of targeting certain cells. See, e.g., Zhang et al., Cancer Metastasis Rev. 15:385-401 (1996). Chase et al., (Nature Biotechnol. 16:444-448 (1998)) used a herpes virus with an inactivated viral ribonucleotide reductase gene that selectively delivered P450 2B1 to tumor cells that overexpress the mammalian ribonucleotide reductase enzyme, which is required for this modified virus to replicate.

In one embodiment, a single viral vector is used to carry both the therapeutic protein encoding gene and the IAP encoding gene. In another embodiment, two viral vectors are used; one carrying the prodrug activating enzyme gene and the other carrying the IAP gene. If two viral vectors are used, they can be derived from the same or a different type of virus, and can be administered simultaneously or sequentially (i.e., without regard for a specific order).

The therapeutic protein encoding gene and the IAP encoding gene can also be delivered using non-viral methods for gene transfer, preferably those whose use in gene therapy is known in the art (Nakanishi, Crit. Rev. Therapeu. Drug Carrier Systems 12:263-310 (1995); Abdallah, et al., Biol Cell 85:1-7 (1995); Zhang et al., Cancer Metastasis Rev. 15:385-401 (1996); Philips, Biologicals 23:13-16 (1995); Lee and Huang, Crit. Rev. Ther. Drug Carrier Syst. 14:173-206 (1997)). Examples of such non-viral vectors for gene delivery include prokaryotic vectors, such as tumor targeted bacterial vectors (Pawelek et al., Cancer Res. 57:4537-4544 (1997)), cationic liposomes, DNA-protein complexes, non-viral T7 autogene vectors (Chen et al., Hum. Gene Ther. 9:729-736 (1998)), fusogenic liposomes, direct injection of nucleic acid (“naked DNA”), particle or receptor-mediated gene transfer, hybrid vectors such as DNA-adenovirus conjugates or other molecular conjugates involving a non-viral and viral component, starburstpolyamidoamine dendrimers (Kukowska-Latallo et al., Proc Natl Acad Sci USA 93:48974902 (1996); Tang et al., Bioconjug. Chem. 7:703-714 (1996)), cationic peptides (Wyman et al., Biochemistry 36:3008-3017 (1997)), and mammalian artificial chromosomes (Ascenzioni et al., Cancer Lett. 118:135-142 (1997)).

In addition, the present invention provides an embodiment of the foregoing methods wherein the therapeutic gene and the IAP gene are delivered using any cellular vector, preferably one whose use for gene therapy is well-established for those skilled in the art. Examples of such cellular vectors for gene therapy include endothelial cells (Rancourt et al., Clin. Cancer Res. 4:265-270 (1998); Qjeifo et al., Cytokines Mol. Ther. 2:89-101 (1996)), macrophages including tumor-infiltrating macrophages (Zufferey et al., Nat. Biotechnol. 15:871-875 (1997); Naldini et al., Science 272:263-267 (1996)), and microencapsulated cells, or tissue engineered constructs, each of which may be modified using viral or non-Viral vectors to carry the prodrug activating enzyme gene and/or the IAP gene, and thus express the prodrug activating enzyme and IAP gene products. Other suitable non-viral vectors will be readily apparent to the skilled artisan. For example, tissue engineered constructs comprise a biocompatible polymer formed into a scaffold suitable for cell growth onto which cells producing a therapeutic protein and IAP engoding gene(s) can be added. Such constructs are well known in the art (see, e.g., WO02/035992, U.S. Pat. Nos. 6,479,064, 6,461,628).

If a single vector is used, the therapeutic gene and the IAP gene can be delivered as a fusion gene which encodes a prodrug activating enzyme-IAP fusion protein. The construction of such a fusion gene is well-established for those skilled in the art (Fisher et al., Methods Enzymol. 272:15-25 (1996); Shet et al., Proc. Natl. Acad. Sci. USA 90:11748-11752 (1993); Fisher et al., Proc. Natl. Acad. Sci. USA 89:10817-10821 (1992); Yabusaki, Biochimie 77:594-603 (1995)), and may allow for highly efficient expression of P450 and IAP activity.

Alternatively, therapeutic gene delivery can be enhanced by including an internal ribosome entry site (IRES) sequence to achieve coordinate expression of the prodrug activating enzyme gene and the IAP gene on a bicistronic message. IRESs of diverse sequence are known. Classic IRESs are nucleic acid sequences containing 500-600 bp that are typical of the 5′ nontransduced regions of picornaviruses, including the polio- and encephalomyocarditis viruses (EMCV). See, e.g., Ghattas et al., Molecular and Cellular Biology 11:5848-5859 (1991); Morgan et al., Nucleic Acids Research 20:1293-1299 (1992). This approach has been used for efficient retroviral coexpression of the two subunits of interleukin-12 (Tahara et al., J. Immunol. 154:6466-6474 (1995)). Another alternative is for the vector to contain both the prodrug activating enzyme gene and the IAP gene under the control of distinct promoters.

In another embodiment of the invention, the preferred therapeutic gene and the IAP gene can be combined with other established prodrug activation gene therapies, including those based on the prodrug activating gene HSV-TK in combination with ganciclovir (GCV) treatment (Moolten, Cancer Gene Therapy 1:279-287 (1994) and the prodrug activating gene cytosine deaminase (CD) in combination with 5-fluorocytosine (5-FC) treatment. 5-Fluorouracil (5-FU), the active metabolite of 5-FC, initiates a caspase 8-dependent apoptotic death (Adachi Y, et al. (1999) Int J Oncol 15: 1191-1196; Aota et al., (2000) Biochem Biophys Res Commun 273: 1168-1174; Ohtani et al. (2000) Anticancer Res 20: 3117-3121), while GCV triphosphate, the active metabolite of GCV, induces caspase 8-mediated cell death in tumor cells that have a functional p53 (Beltinger C, et al. (1999) Proc Natl Acad Sci USA 96: 8699-8704), and caspase 9-dependent cell death in p53-deficient cells (Tomicic M T, et al. (2002) Oncogene 21: 2141-2153). Expression of apoptosis inhibiting agents will prolong the life of cells transduced with these prodrug activating genes according to the present invention, and this will to an increased bystander activity. In the case of CD, transduced ‘5-FU factory’ tumor cells display up to a 500-fold greater susceptibility to killing than do bystander tumor cells (Lawrence T S, et al. (1998) Cancer Res 58: 2588-2593), highlighting the need to delay the death of these cells. This can be accomplished either by separate transfer of both suicide genes (Aghi et al., J. Natl. Cancer Inst. 90:370-380 (1998); Uckert et al., Human Gene Therapy 9:855-865(1998)), or by transfer of a fusion gene encoding both drug activation enzymes (Rogulsli et al., Human Gene Therapy 8:73-85 (1997)). Therapeutic gene/LAP gene therapy may also be combined with other established cancer therapeutic genes, including cytokines, such as interleukin 2 (Clary et al., Cancer Gene Ther. 4:97-104 (1997); O'Malley et al., Ann. N.Y Acad. Sci. 842:163-170 (1998)), interleukin 4 (Benedetti et al., Human Gene Therapy 8:1345-1353 (1997)), and interleukin 12 (Chen et al., Immunol. 159:351-359 (1997)).

In an additional embodiment, the targeting specificity for therapeutic gene and LAP gene delivery may be facilitated by targeted delivery or targeted expression (“transcriptional targeting”), including the use of tumor-specific or tumor-selective DNA enhancer sequences to provide for selective replication of a viral vector (Li et al, Cancer Research 61: 6428-6436 (2002)) or to selectively activate expression of the transduced gene in the tumor cell at either the primary tumor site or its metastases (Miller and Whelan, Hum. Gene Ther. 8:803-815 (1997); Walther and Stein, J. Mol. Med. 74:379-392 (1996); Schnierle and Groner, Gene Therapy 3:1069-1073 (1996); Lan, K -H., et al., Cancer Res. 57:4279-4284 (1997)); Dachs, G. U., et al., Oncol. Res. 9:313-325 (1997)). Examples of this approach include those DNA enhancers that have been derived from genes that encode tyrosinase (allowing for targeting to melanoma), ERBB2 (targeting to pancreatic cancer), carcinoembryonic antigen (targeting to lung and gastrointestinal malignancies, including colon, pancreatic and gastric cancer), DF3/MUC 1 (targeting to breast cancer), and alpha-fetoprotein (targeting to hepatoma). DNA enhancers derived from genes that are preferentially expressed in tumor-associated endothelial cells can be used in a similar way to achieve selective expression of the therapeutic gene and the LAP within the tumor vasculature. The use of synthetic gene regulation systems, which allow for transcriptional control and other forms of regulated expression of the prodrug activating enzyme and/or IAP genes, may also be used (Miller and Whelan, Hum. Gene Ther. 8:803-815 (1997); Vile, Semin. Cancer Biol. 5:429-436 (1994); Hwang et al., J. Virol. 70:8138-8141 (1996); Massie et al., J. Virol. 72:2289-2296 (1998)).

In another embodiment, the invention provides a method for enhancing vector spread. A replicating vector carrying a heterologous gene may be used to deliver the apoptosis inhibiting agent, which suppresses host cell apoptosis resulting in increased vector spread and hence increased expression of the heterologous gene. For example, the replicating vector may itself have intrinsic therapeutic activity, such as in the case of tumor cell replicating, oncolytic viral vectors, such as E1b region 55 kd gene-deleted adenoviral vectors, whose spread may be increased to encompass a greater fraction of the target tumor cells by introduction of an apoptosis inhibiting agent according to the present invention. Alternatively, the apoptosis inhibiting agent may be delivered via a non-replicating vector, which may also deliver a desired heterologous gene; in that case the non-replicating vector is administered in combination with (either before, after or simultaneously) with a replicating virus.

The mammal treated using the methods of the present invention is preferably human.

The cells that can be treated using the methods of the present invention include neoplastic cells such as solid tumors, including brain, lung, colon, breast, ovarian, prostate and liver cancers, melanomas, soft tissue sarcomas and hematopoietic cancers, including leukemias, as well as tumor-associated cells, including but not limited to tumor-associated endothelial cells. Preferably the cell is a solid tumor cell or a tumor-associated endothelial cell.

The term “chemotherapeutic agent or drug that is activated by the product of a prodrug-activating gene” is meant to include any pharmaceutical agent that can be used in the treatment of neoplasms, and that is capable of being activated by an enzyme-catalyzed process.

The terms “activating”, “bioactivating” or “activating”, a chemotherapeutic agent, are used interchangeably herein, and are meant to describe any metabolic reaction that increases the cytotoxic or cytostatic activity or otherwise increases the therapeutic efficacy of the agent; or that confers on the agent an additional mechanism of action beyond that which the agent exhibits in the absence of the metabolic reaction. By “cytotoxic” or “cytostatic” is intended causing or leading to cell death or slower cell growth. Examples of P450-activated chemotherapeutic agents are CPA, ifosfamide, dacarbazine, procarbazine, thio-TEPA, etoposide, 4-ipomeanol, 2-aminoanthracene, tamoxifen, methoxymorpholinyl doxorubicin, tegafur, acetaminophen as well as various cyclic phosph(on)ate ester-containing P450 prodrugs. Other P450-activated chemotherapeutic agents, and chemotherapeutic agents activated by other prodrug-activating enzymes are known to those skilled in the art and can also be used in the present invention.

Treating said neoplastic cells with a chemotherapeutic agent is intended to include both the local delivery of the prodrug into or near the site of the tumor by, e.g., slow-release pellets or catheter, as well as the systemic administration of the chemotherapeutic agent, i.e., through intraperitoneal, intravenous, parenteral, or intramuscular routes. Localized delivery of the drug is expected to increase the fraction of the drug activated within the tumor, and thus increase drug efficacy and is therefore preferred.

Dosages of a particular chemotherapeutic agent may be administered according to current standard clinical practice. See, e.g., Hubbard, S. M. and Jenkins, J. F., “Chemotherapy Administration: Practical Guidelines” in Cancer Chemotherapy: Principles and Practice, Chabner and Collins, eds., J. B. Lippincott Company, Philadelphia, Pa. (1990), pages 449463. For example, standard clinical dosages for CPA in adults range from approximately 600-1000 mg/meter.sup.2 (m.sup.2) (Struck, R. F., et al., Cancer Research 47:2723-2726 (1987); standard daily dosages for IFA range from approximately 1 to 3 grams/m.sup.2 (Kurowski, V. and Wagner, T., Cancer Chemother. Pharmacol. 33:3642 (1993)). Standard clinical practice may involve body surface area (BSA)-based dose calculations, as well as individualization of dosages based on pharmacokinetic optimization using plasma drug and metabolite concentrations (“therapeutic drug monitoring” or TDM). Such concentrations may be obtained using limited sampling or other pharmacokinetic sampling and modeling techniques (van Warmerdam, L. J., et al., Neth J. Med. 51:30-35 (1997); Desoize, B. and Robert, J., Eur. J. Cancer 30A:844-851 (1994); Gurney, H., J. Clin. Oncol. 14:2590-2611′ (1996)). Other factors, known to those skilled in the art, such as the clinical status and age of the patient, will also contribute to dosage adjustment.

We have discovered that the inhibition of caspase activity by IAPs in a cell expressing a prodrug activation gene will not block stress-induced, mitochondrial-dependent cell death. Therefore, while the overexpression of IAPs in tumor cells may lead to slower tumor cell death, it is unlikely to block tumor cell death induced by chemotherapeutic drugs, such as CPA, whose action is mediated by the mitochondrial cell death pathway.

CPA activates a mitochondrial-regulated pathway of cell death, with anti-apoptotic factors, such as the IAP p35 conferring prolonged cell death to a tumor cell transduced with a prodrug activation gene but without enhanced tumor cell survival. This finding raises the possibility of using IAPs to modulate the rate of CPA-induced cell death. The discovery is based upon the observation that in cells undergoing stress- or DNA damage-induced cell death, IAPs block caspase activation downstream of the mitochondrial transition and therefore do not interfere with the release of cytochrome C mediated by pro-apoptotic Bcl-2 family members, such as Bax (Roy et al., Embo J. 16:6914-6925 (1997)). Consequently, even in the presence of IAPs, cell death ultimately occurs, once the mitochondrial apoptotic pathway has been initiated and mitochondrial potential is lost, regardless of the status of caspase activation (Reed, Apoptosis and Cancer Chemotherapy 99-116 (1999)).

Thus, tumor cells transduced with a prodrug-activating P450 gene and with a caspase inhibitor and anti-apoptotic factor, such as p35, displayed a striking increase in their potential for prolonged prodrug activation. This effect was found to be greatest in tumor cells exposed to the P450 prodrug CPA for an 8 hr period, designed to model the effective time period of drug exposure in bolus CPA-treated patients in vivo. Tumor cells expressing p35 in conjunction with P450 gene therapy are not drug resistant, however, as indicated by the absence of long-term tumor cell survival or colony formation activity following CPA treatment. These findings demonstrate that anti-apoptotic factors can be employed in a novel way to enhance prodrug activation gene therapy, by delaying cell death in a manner that increases the net production of bystander cytotoxic metabolites, and hence the overall therapeutic effectiveness of the GDEPT strategy.

Cellular damage, including DNA damage induced by cancer chemotherapeutic drugs, induces tumor cell death by apoptosis. Apoptosis is triggered by a complex cascade of events, central to which is the activation of caspases, cysteine proteases that include both upstream (initiator) and downstream (effector) caspases (Nunez et al., Oncogene. 17:3237-3245 (1998)). Two major pathways of caspase-dependent apoptosis have been identified. One pathway is initiated by the formation of a cell death-inducing plasma membrane receptor signaling complex (Scaffidi et al., J Biol. Chem. 274:22532-22538 (1999)), which induces aggregation and activation of the initiator caspase 8 (Ashkenazi and Dixit, Science. 281:1305-1308 (1998); Green, Cell. 94:695-698 (1998); Micheau et al., J Biol. Chem. 274:7987-7992 (1999)). A second apoptotic pathway is triggered by cellular stress such as DNA damage (Green, Cell. 94:695-698 (1998); Sun et al., J Biol. Chem. 274:5053-5060 (1999)) and is primarily associated with a cell death pathway linked to the release of pro-apoptotic molecules from the mitochondria (mitochondrial transition) and the subsequent activation of a distinct initiator caspase, caspase 9 (Green and Reed, Science. 281:1309-1312 (1998); Li et al., Cell. 91:479489 (1997); Susin et al., J Exp Med. 189:381-394 (1999)). Once activated, caspase 8 and caspase 9 cleave and thereby activate downstream caspase family members, such as caspases 3 and 7 (Slee et al., J. Cell Biol. 144:281-292 (1999)). These downstream effector caspases, in turn, cleave multiple cellular proteins, triggering the phenotypic changes that are associated with apoptosis. Previous studies demonstrate that the mitochondrial-mediated caspase 9 pathway is the key regulatory pathway responsible for CPA-induced tumor cell death (Schwartz and Waxman, Mol Pharmacol. 60:1268-1279 (2001)).

The expression in tumor cells of pro-apoptotic factors, including Bax, p53, Trail, various caspases, and pro-apoptotic factors belonging to the Bcl-2 family, enhances killing of tumor cells and has been employed in both preclinical and clinical anti-cancer gene therapies, either alone or in combination with traditional chemotherapy (Kagawa et al., Cancer Res. 60:1157-1161 (2000); Kagawa et al., Cancer Res. 61:3330-3338 (2001); Komata et al., Cancer Res. 61:5796-5802 (2001); Rizk et al., Cancer Gene Ther. 6:291-301 (1999); Xie et al., Cancer Res. 61:6795-6804 (2001)). This approach is based on the finding that pro-apoptotic factors augment the cytotoxic effects of many chemotherapeutic drugs by lowering the threshold drug level required to induce tumor cell death. In the case of CPA and other alkylating agents, enhanced cytotoxicity can be achieved by other approaches, including depletion of protective small molecules, such as glutathione (GSH), or by decreasing the expression or activity of protective enzymes, such as glutathione S-transferases (GSTs) (Chen and Waxman, Biochem Pharmacol. 47:1079-1087 (1994); Ozols et al., Biochem Pharmacol. 36:147-153 (1987); Waxman, Cancer Res. 50:6449-6454 (1990)), and in the case of CPA, aldehyde dehydrogenases, which detoxify the activated form of CPA (Russo et al., Prog Clin Biol Res. 290:65-79 (1989); Sahovic et al., Cancer Res. 48: 1223-1226 (1988); Sladek, Curr Pharm Des. 5:607-625 (1999)). However, an important limitation of the expression of pro-apoptotic factors or other chemosensitization factors that is highlighted by our findings is that it leads to more rapid death of the factory cell, and thus cannot be combined in a useful manner with a gene therapy approach to cancer treatment that is reliant on a prodrug-activation enzyme or a soluble, or secretable therapeutic factor.

It is to be understood that while the invention has been described in conjunction with the preferred specific embodiments thereof that the foregoing description as well as the examples that follow are intended to illustrate and not limit the scope of the invention. Other aspects, advantages and modification within the scope of the invention will be apparent to those skilled in the art to which the invention pertains.

### EXAMPLES

Generation of Stable Cell Lines: Rat 9L/P450 gliosarcoma cells and derivatives, all prepared by retroviral transduction, were grown as monolayers cultured at 37° C. in a humidified atmosphere containing 5% CO₂ in Dulbecco's modified Eagle's medium (DMEM) containing 10% fetal bovine serum. The 9L/P450 cells used in this study were kindly provided by Dr. Y. Jounaidi of this laboratory. These cells co-express human P450 2B6 and human P450 reductase and correspond to the 9L/2B6/Red cells described previously (Jounaidi and Waxman, Cancer Research. 61:4437-4444 (2001)). 9L/P450 cells expressing IAPs (i.e., p35, hIAP1, hIAP2 or Survivin) were generated by retroviral transduction. p35 cDNA (Clem et al., Science. 254:1388-1390 (1991)) was obtained from Dr. Tom Gilmore (Boston University, Boston, Mass.) and subcloned by blunt end ligation from the plasmid pBluescript/p35 into the HindIII and ClaI sites of the retroviral plasmid pLNCX (Clontech Laboratories, Palo Alto, Calif.). HIAP1 and hIAP2 cDNAs, both myc-tagged and cloned into pcDNA3 (Roy et al., Embo J. 16:6914-6925 (1997)) were obtained from Dr. E. LaCasse, Apoptogen Inc. (Ottawa, Ontario, Canada) and subcloned by directional ligation into the HindIII and ClaI sites of the retroviral plasmid pLNCX. Survivin cDNA in pcDNA3 (Ambrosini et al., Nat Med. 3:917-921 (1997)) was obtained from Dr. D. C. Altieri (Boyer Center for Molecular Medicine, Yale University, New Haven, Conn.) and was also subcloned by directional ligation into the HindIII and ClaI sites of pLNCX. The generation of retroviral particles was described previously (Schwartz and Waxman, Mol Pharmacol. 60:1268-1279 (2001)). Briefly, the packaging cell line Bosc 23 (Pear et al., Proc Natl Acad Sci USA. 90:8392-8396 (1993)) was transfected with pLNCX vectors containing each of the IAP cDNAs. Media containing retroviral particles was collected 48 hr later and placed on 9L/P450 cells (Hecht et al., Gene Therapy of Cancer 85-94 (2000)). The cells were incubated for 48 hr and subsequently selected with 1.5 mg/ml G418 for 5 days. The resultant pools of transduced cells were shown to stably express the IAP, as shown by RT-PCR (p35 and Survivin). To obtain clonal 9L/P450/p35 cell lines, twelve individual p35-expressing colonies were isolated from the original pool of retrovirally transduced cells by plating the cells at a calculated density of 1 cell per well in a 96 well plate. Individual clones were assayed for 4-OH-CPA production and caspase 3 activity following CPA treatment. Four colonies that were shown to have reduced caspase 3 activity and yet retained substantial CPA 4-hydroxylase activity were chosen for more detailed analysis. P450 2B6 expression was evaluated by Western blotting and P450 reductase activity was assayed by cytochrome C reduction (both assayed in whole cell extracts). p35 expression was confirmed by RT-PCR (see below).

RT-PCR: 9L/P450, 9L/P450/p35 and 9L/P450/Survivin cells were plated at 2×10⁵ cells in 35 mm dishes. RNA isolation was carried out using Trizol reagent (GIBCO BRL, Gaithersburg, Md., Cat # 15596). One ml of Trizol was added to each 35 mm dish, and the cell lysate was passed several times through a pipette tip. The homogenate was incubated for 5 min and transferred to a clean tube. Chloroform (200 μl) was added to each tube, the samples were mixed, incubated for 3 min, and centrifuged at 11,000×g for 15 min at 4° C. The aqueous phase was transferred to a clean tube and the RNA was precipitated by incubation with 0.5 ml of isopropyl alcohol for 10 min followed by centrifugation at 11,000×g for 10 min at 4° C. The RNA pellet was washed once with cold 75% ethanol in DEPC-treated water and centrifuged at 7,000×g for 5 min at 4° C. The pellet was resuspended in 50 μl DEPC-treated water and incubated for 10 min at 60° C. First strand synthesis was performed using the SuperScript preamplification system (GIBCO BRL, Gaithersburg, Md., Cat # 18089-011). Total RNA (2 μg) was mixed with 0.5 μg of Oligo(dT)₁₂₋₁₈ and then incubated at 70° C. for 10 min. The reaction mix was then cooled to room temperature followed by addition of 50 μl of 10× PCR buffer, 25 mM MgCl₂, 10 mM dNTP mix, and 0.1 M DTT. The total mix was incubated at 42° C. for 5 min and then 200 units of SuperScript II reverse transcriptase was added to each tube and incubated for an additional 50 min. The reaction was terminated by incubation at 70° C. for 15 min. Samples were then incubated for 20 min at 37° C. with RNase H (USB, Cleveland, Ohio). PCR was performed using 5 μl of the RNase H-digested first strand synthesis product using 50 μl of 10× PCR buffer, 25 mM MgCl₂, 10 mM dNTP mix, Taq polymerase, and 10 μM gene-specific primers (for p35 or Survivin). Samples were incubated at 94° C. for 3 min, then subjected to 35 cycles of denaturation at 94° C. for 1 min, annealing at 56° C. for 1 min and extension at 72° C. for 1 min. The expected PCR product lengths were 561 bp (p35-specific primers: 5′-CCTCCCGCTGTTTGACCTCCTA-3′ [SEQ ID NO: 1] and 5′-ATCCCGGCTTCAACACGCATACC-3′ [SEQ ID NO: 2]) and 614 bp (Survivin-specific primers: 5′-GTGGGCCCCTTAGCAATGTCTTAG-3′ [SEQ ID NO: 3] and 5′-CACCCCGTTTCCCCAATGA-3′ [SEQ ID NO: 4]).

Western Blotting: 9L/P450 cell extracts prepared in lysis buffer (20% glycerol, 1% Trition X-100, 20 mM Hepes pH 7.9, 1 mM EDTA, 1 mM EGTA, 20 mM NaF, 1 mM Na₄P₂O₇, 1 mM DTT, 1 mM Na₃VO₄, 1 mg/ml leupeptin, 1 mg/ml pepstatin) were analyzed by Western blotting as detailed previously (Schwartz and Waxman, Mol Pharmacol. 60:1268-1279. (2001)). P450 2B6 protein was detected using monoclonal anti-P450 2B6 antibody at a dilution of 1:5000 (Gentest Corp. Cat. # A326, Woburn, Mass.). P450 reductase was detected with an anti-peptide antibody (1:5000 dilution) obtained from Dr. R. Edwards (Royal Postgraduate Medical School, London, United Kingdom). After washing, the blots were incubated with goat anti-mouse HRP-linked or goat anti-rabbit secondary antibody (Pierce, Rockford, Ill., cat #31430 and #31460, respectively). Blots were visualized with ECL Western blotting detection reagent (Amersham Pharmacia Biotech; Cat. # RPN2106) and exposed to Kodak X-OMAT blue film XB-1.

Growth Inhibition Assay: 9L/P450 and 9L/P450-derived cells lines were plated at 1.5×10⁵ cells per well in 12 well dishes in 1.5 ml media unless indicated otherwise. Cells were grown overnight and then treated with culture media containing 1 mM CPA. Cells remaining on the plate at the indicated times were washed twice with cold PBS and then stained with crystal violet solution, as described previously (Schwartz and Waxman, Mol Pharmacol. 60:1268-1279. (2001)). The stain was eluted from the cells and the absorbance read at 595 nm to determine the relative protein content of each sample (Jounaidi et al., Cancer Res. 58:4391-4401 (1998)).

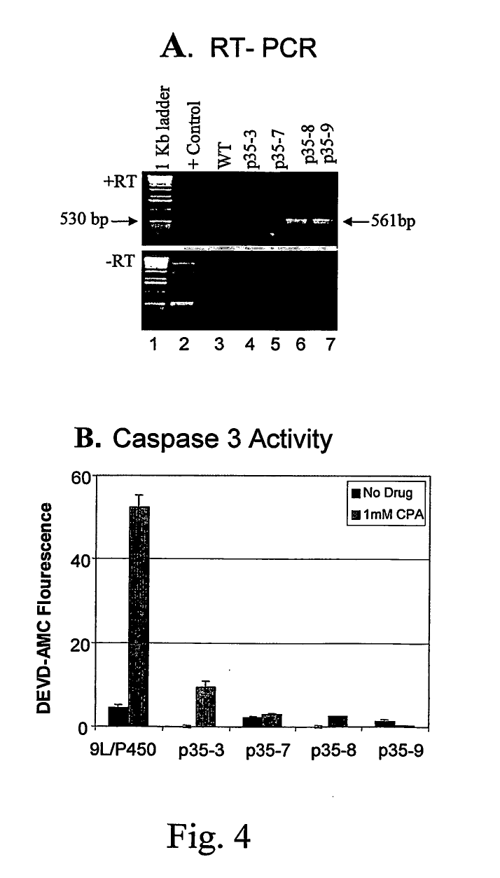

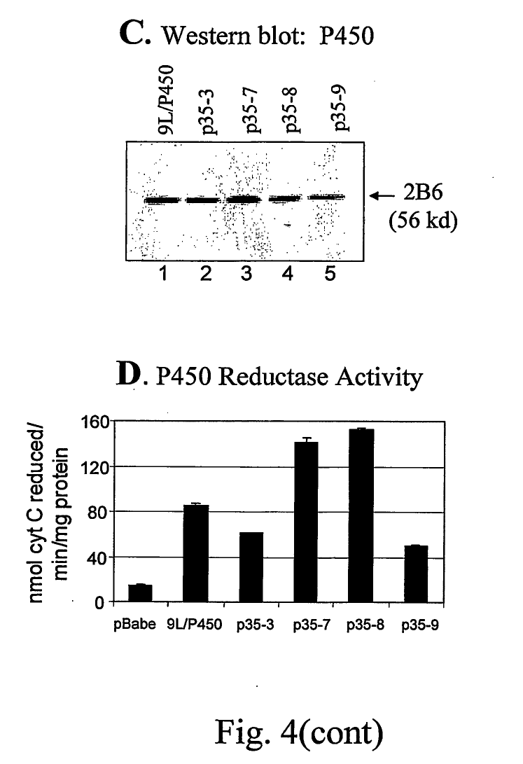

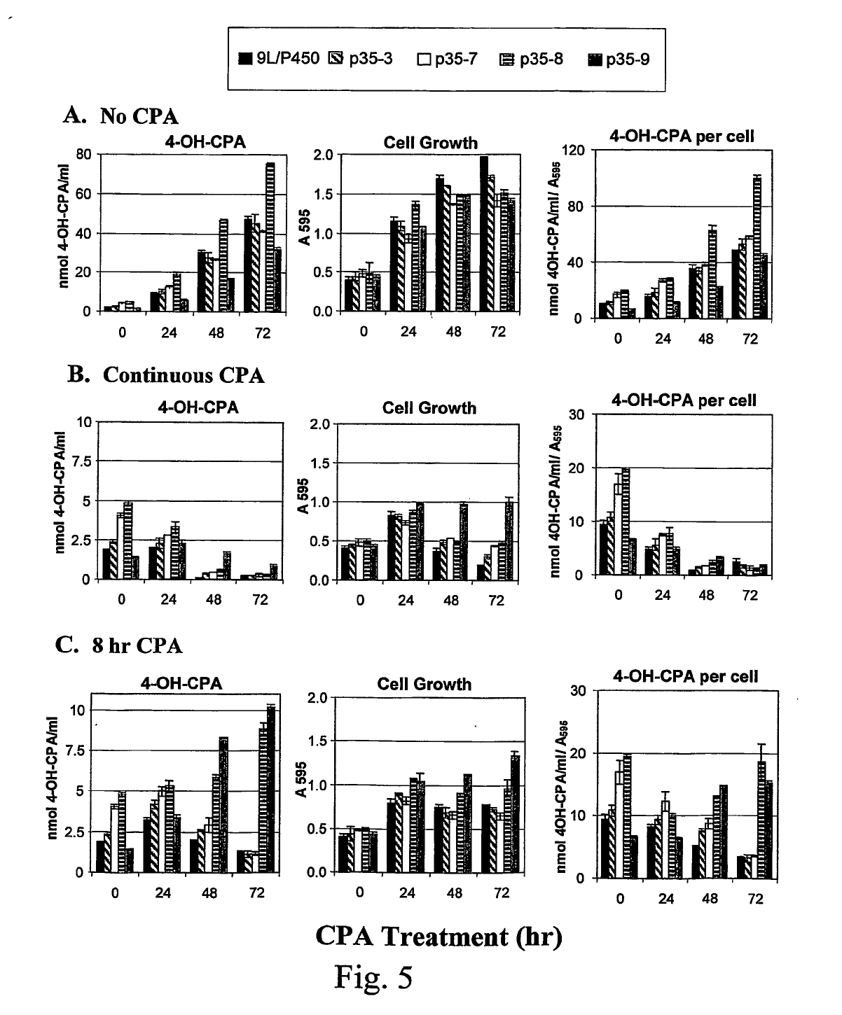

Bystander Experiments. Two bystander experiment protocols were used. In protocol 1 (direct bystander cell contact protocol), 9L/LacZ cells (bystander cells) were co-cultured with either 9L/P450 or 9L/P450/p35-9 cells. In protocol 2 (media contact bystander protocol), the bystander cells were separated from the P450-expressing 9L cells by a permeable membrane. For protocol 1, 9L/P450 and 9L/P450/p35-9 cells were plated in triplicate at 0.5×10⁵ and 0.75×10⁵ cells per well, respectively, in 12 well tissue culture dishes containing 1.5 ml of culture media per well. The higher number of 9L/P450/p35-9 cells compensates for the lower intrinsic CPA 4-hydroxylase activity of this cell line compared to 9L/P450 cells (see FIG. 4 and FIG. 5, below). Twenty-four hr after seeding, the cells were treated with 1 mM CPA for 8 hr. Forty-eight hr after beginning the first drug treatment, 9L/LacZ bystander cells were plated on top of the 9L/P450 and 9L/P450/p35-9 cell monolayers at 2.5×10⁵ 9L/LacZ cells per well. Twenty-four hr later the mixed cell populations were treated with 0, 0.125, 0.25 or 0.5 mM CPA for a second 8 hr drug treatment period. The cells were then grown in drug-free media for an additional 16 hr (i.e., 24 hr from the start of the second drug treatment), at which point the cells were trypsinized, collected, counted and replated at various densities in 6 well plates to allow for the growth of individual colonies, as follows: Drug-free control cells were replated in duplicate at 100, 150 and 250 cells/well. CPA-treated 9L/P450+9L/LacZ cells were replated as follows: 500, 1000 and 2000 cells/well (0.125 mM CPA-treated samples); 1000, 2000 and 4000 cells/well (0.25 mM CPA-treated samples); and 2000, 5000 and 10000 cells/well (0.5 mM CPA-treated samples). CPA-treated 9L/P450/p35-9+9L/LacZ cells were replated at twice the densities shown above. Cells were grown for 10-11 days, with media changes every 3 days, and then stained with X-gal. LacZ-positive colonies containing≧50 cells were counted. Cells were then re-stained with crystal violet and the total number of colonies was determined. The number of 9L/P450 or 9L/P450/p35-9 colonies on each plate was then calculated by subtracting number of the Lac Z-positive colonies from each sample. The colony efficiency (number of colonies/number of cells seeded) was then calculated for each treatment group.

For protocol 2, 9L/P450 and 9L/P450/p35-9 cells were plated at 0.6×10⁵ cells and 0.9×10⁵ cells per well, respectively, on cell culture inserts (0.4 μM PET Track-etched membrane; B-D Labware, cat. # 3090, Franklin Lakes, N.J.). The inserts were suspended in 6 well tissue culture dishes containing 3 ml media/well. Twenty-four hr after plating the cells were treated with 1 mM CPA for 8 hr. Forty-eight hr after beginning the first drug treatment, 9L/LacZ cells were plated in a separate set of 6 well tissue dishes at 6×10⁵ cells per well. Four hr later the inserts containing 9L/P450 or 9L/P450/p35-9 cells were placed into the 9L/LacZ-seeded wells. Twenty-four hr after plating the 9L/LacZ cells, the wells (containing 9L/LacZ+9L/P450 or 9L/LacZ+9L/P450/p35-9 cells) were treated with 0, 0.125, 0.25 or 0.5 mM CPA for an 8 hr period. The drug-treated cells were then grown in drug-free media for 16 hr, at which point the two populations of cells in each well (i.e., 9L/Lac Z (lower chamber) and 9L/P450 (or 9L/P450/p35-9) cells (upper chamber)) were trypsinized, collected separately, counted and replated at various densities in 6 well dishes for a colony formation assay as described for protocol 1, but at the following cell densities: drug-free control cells were replated in duplicate at 100, 150 and 250 cells/well; 9L/LacZ+9L/P450 samples at 250, 500 and 1000 cells/well (0.125 mM CPA), 500, 1000 and 2000 cells/well (0.25 mM CPA) or 1000, 2000 and 5000 cells/well (0.5 mM CPA). The 9L/LacZ+9L/P450/p35-9 samples were replated at 2000, 4000 and 7000 cells/well (0.125 mM CPA), 5000, 10000 and 20000 cells/well (0.25 mM CPA) and 10000, 20000 and 40000 cells/well (0.5 mM CPA). Cells were grown for 10-11 days, stained with crystal violet and colony formation then determined.

Caspase Activity Measurements: Caspase activities were assayed as described previously (Schwartz and Waxman, Mol Pharmacol. 60:1268-1279. (2001)). Briefly, 9L/P450 and 9L/P450-derived cells were treated with CPA for the times indicated in each experiment. Floating and attached cells were collected, pooled, dissolved in lysis buffer (10 mM Hepes buffer (pH 7.4) containing 2 mM EDTA, 0.1% CHAPS detergent, 5 mM DTT, 350 ng/ml PMSF, 10 ng/ml pepstatin A, 10 ng/ml aprotinin, and 20 ng/ml leupeptin) and then subjected to three freeze-thaw cycles. Lysates were spun and the supernatant (cell extract) was assayed for caspase 3, 8 and 9 activities by incubation with caspase form-selective substrates: Ac-DEVD-AMC (Biomol; cat # P-411) for caspase 3 (Talanian et al., J Biol. Chem. 272:9677-9682 (1997); Thomberry et al., J Biol. Chem. 272:17907-17911 (1997)), LETD-AFC (BioRad; cat# 170-3186) for caspase 8 and Ac-LEHD-AFC (BioRad; cat# 170-3190) for caspase 9. Assays were carried out at 37° C. for 15 min (caspase 3) or 3 hr (caspases 8 and 9), and background activities was determined for each sample by pre-incubation with or without caspase form-selective inhibitors as described previously (Schwartz and Waxman, Mol Pharmacol. 60:1268-1279 (2001)).

Quantitation of Cellular 4-OH-CPA Production: 9L/P450 cells and 9L/P450-derived cell lines were plated in 12 well tissue culture plates at 1.5×10⁵ cells per well with 1.5 ml per well. Twenty four hr later the cells were treated with media containing 1 mM CPA. To determine cellular CPA 4-hydroxylase activity, cells were incubated at the times indicated in each figure for a 4 hr period with fresh media containing 1 mM CPA. Semicarbazide (5 mM) was included in the media to trap and stabilize the initial 4-OH-CPA metabolite. An aliquot of media (0.5 ml) was removed from each well and snap frozen in liquid nitrogen. Cells remaining on the plate were washed with 1× PBS and stained with crystal violet (A₅₉₅). Protein in the culture media was then precipitated by the sequential addition of 200 μl of ice-cold ZnSO₄, 200 μl of saturated barium hydroxide, and 100 μl of 0.01 M HCl. Samples were spun at 14,000 rpm for 20 min and 750 μl of the supernatant was transferred to a new tube. Acrolein was derivatized with 3-aminophenol by the addition of 400 μl of fluorescent reagent (6 mg 3-aminophenol mixed with 6 mg hydroxylamine hydrochloride in 1 ml of 1 N HCl). The samples were heated at 90° C. for 20 min in the dark, cooled to room temperature and analyzed (100 μl) on a C18 HPLC column (Huang and Waxman, Anal Biochem. 273:117-125 (1999)). Calibration curves were generated using 4OOH-CPA in cell culture media. Cellular CPA 4-hydroxylase activity was then calculated (nmol 4-OH-CPA/ml media/A₅₉₅).

P450 Reductase Activity: Cell extracts were prepared in 5× KPi buffer as described above. P450 reductase activity was assayed using 50 μg of extract protein diluted into 1 ml of cytochrome C buffer (0.526 mg/ml cytochrome C in 0.3 M KPi, pH 7.7). Assays were initiated by addition of 25 μl of 4 mg/ml NADPH. The rate of cytochrome C reduction was monitored at 550 nm over a 3 min period on a Lambda 35 UV/Visible spectrophotometer (Perkin Elmer Instruments, Norwalk Conn.).

Glutathione depletion sensitizes 9L/P450 cells to CPA but decreases net production of 4-OH-CPA. The expression of P450 2B6 substantially enhances the chemosensitivity of tumor cells to P450-activated anti-cancer prodrugs, such as CPA (Chen et al., Cancer Res. 57:4830-4837 (1997); Jounaidi et al., Cancer Res. 58:4391-4401 (1998)). Tumor cells can also be sensitized to CPA using the GSH synthesis inhibitor buthionine sulfoximine (BSO), which depletes intracellular GSH and thereby decreases cellular GST activity, which has been associated with inactivation of, and resistance to 4-OH-CPA and other anticancer alkating agents (Chen and Waxman, Biochem Pharmacol. 47:1079-1087 (1994); Chen and Waxman, Biochem Pharmacol. 49:1691-1701 (1995)).

We therefore anticipated that the depletion of GSH in P450-expressing tumor cells by BSO pretreatment (50 μM for 24 hr) would 1) decrease the inactivation of 4-OH-CPA by cellular GSH, thereby increasing intracellular 4-OH-CPA levels and 4-OH-CPA released into the culture media, and 2) enhance the cytotoxicity of CPA toward the P450-expressing tumor cells and toward P450-deficient bystander tumor cells.

To test these hypotheses, 9L tumor cells transduced with P450 2B6 were pretreated with BSO to deplete cellular GSH and then exposed to CPA. BSO treatment substantially increased the cell's sensitivity to CPA (1 mM for 72 hr), as indicated by visual examination of the culture (FIG. 1A) and by the BSO-dependent decrease in cell survival, from 62% to 13% (FIG. 1B). Analysis of the CPA 4-hydroxylase (prodrug activation) activity of the 9L/P450 cells revealed, however, that BSO treatment led to an 86% decrease in the cell's capacity to produce and release 4-OH-CPA into the culture media after 24 hr of CPA treatment (FIG. 1C, left). A smaller but still significant decrease (53%) in 4-OH-CPA production was seen in the absence of BSO. These decreases in cellular CPA 4-hydroxylase activity were not observed at earlier time points after CPA addition, and in the case of the BSO-pretreated cells, were associated with an increase in CPA cytotoxicity (FIG. 1C, right). Thus, BSO pretreatment, although effective at increasing the cytotoxicity of CPA to P450-expressing tumor cells, has the surprising and undesirable effect of decreasing the generation of activated prodrug released into the culture media.

Expression of IAPs inhibits caspase activation and desensitizes 9L/P450 cells to CPA-induced cell death. In a typical protocol only a small fraction of the target tumor cell population is successfully transduced by the vector and expresses the therapeutic gene.

Accordingly, in view of our findings in BSO-treated cells (FIG. 1), we hypothesized that in order to achieve the greatest bystander cytotoxic effect, it might be useful to impart transient CPA resistance to those tumor cells that are transduced with the vector and produce activated CPA metabolites. In this way, one would obtain a more efficient prodrug activating ‘factory cell’, i.e., one that produces larger quantities of activated prodrug over an extended period of time before undergoing cell death. We have shown previously that 9L/P450 cells undergo caspase 9-dependent apoptosis following CPA treatment (Schwartz and Waxman, Mol Pharmacol. 60:1268-1279. (2001)).

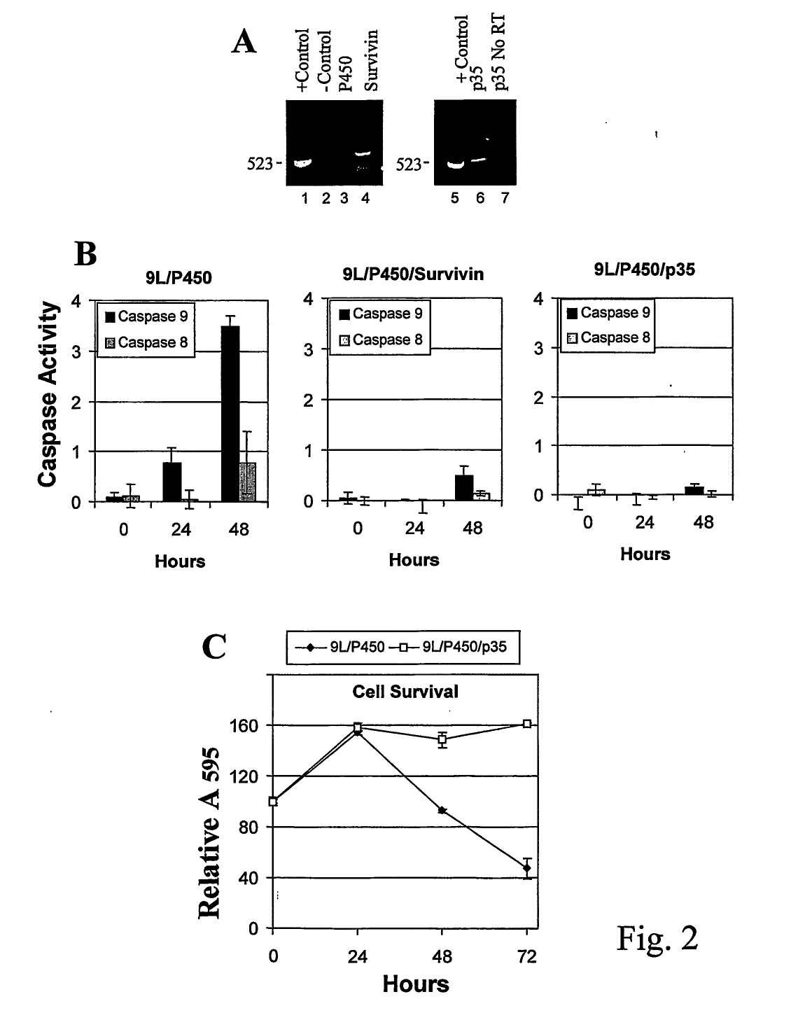

We therefore investigated whether one or more inhibitors of apoptosis (IAPs), which act by inhibiting caspase activity, could be used to prolong the survival of 9L/P450 cells and thereby enable the cells to produce activated CPA metabolites for an extended time period. 9L/P450 cells that express four different IAPs were prepared by retroviral transduction using a protocol that generates a large pool of independent clones, each containing the IAP cDNA at a distinct site of integration (FIG. 2A). Of the four IAPs tested hIAP1, hIAP2, Survivin and p35), Survivin and p35 were the most effective at inhibiting of caspase activity in the CPA-treated 9L/P450 cells (FIG. 2B and data not shown). The expression of p35 resulted in a near-complete block in the induction of caspase activity following CPA treatment and, consequently, p35 was selected for further investigation.

Other studies revealed that the expression of p35 in 9L/P450 cells imparted a cytostatic response to CPA (1 mM for 72 hr); this contrasts to the cytotoxic response seen in the absence of p35 (FIG. 2C). Expression of the anti-apoptotic factor Bcl-2 also changes the cellular response to CPA from a cytotoxic response to a cytostatic response (Schwartz and Waxman, Mol Pharmacol. 60:1268-1279. (2001)).

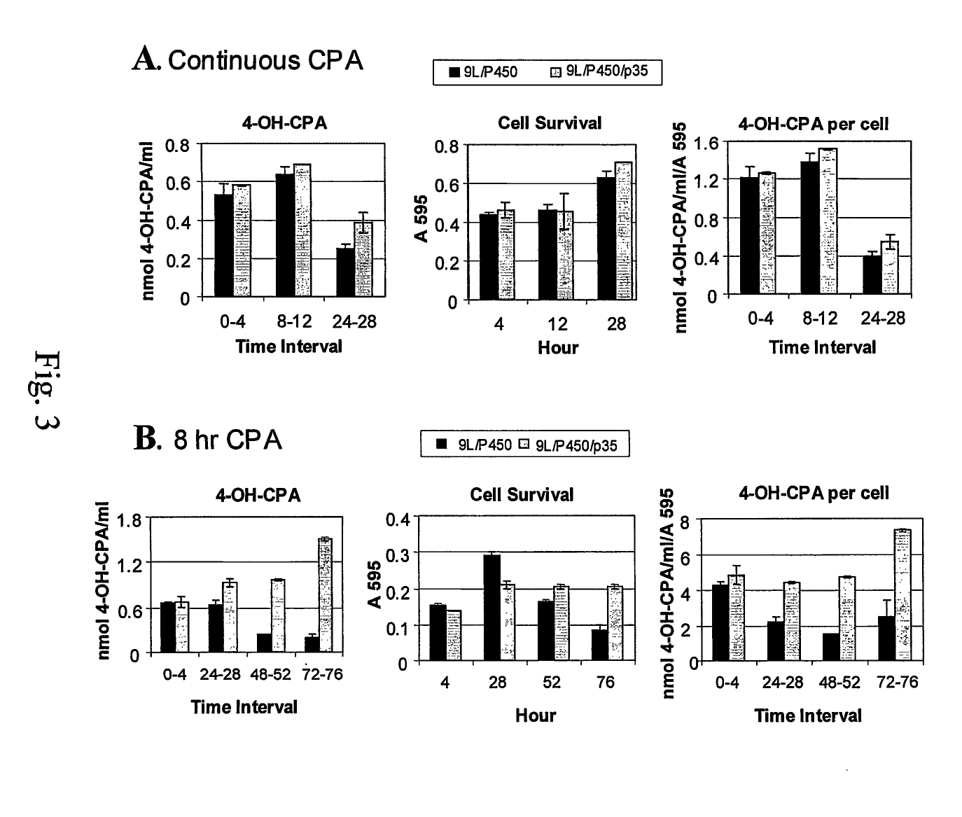

p35 expression sustain the ability of 9L/P450 cells to activate CPA. The prolonged viability of CPA-treated 9L/P450/p35 cells compared to CPA-treated 9L/P450 cells is associated with maintenance of cellular CPA 4-hydroxylase activity for a somewhat longer period of time. This is indicated by the 53% decrease in the capacity of 9L/P450 cells to activate CPA 24 hr after their initial drug exposure, as compared to only a 34% decrease in the rate of CPA activation in 9L/P450/p35 cells under the same conditions (FIG. 3A, left panel). The CPA treatment schedule used in this in vitro experiment, continuous CPA exposure, mimics the continuous 4-day CPA infusion protocol used in patients receiving high dose CPA in combination with autologous bone marrow transplantation (Antman et al., J Clin Oncol. 10:102-110 (1992); Ayash et al., J Clin Oncol. 10:995-1000 (1992)). However, this schedule differs from conventional CPA treatment protocols, where CPA is administered as a bolus and where the half-lives of CPA and its active metabolite phosphoramide mustard in adult patients are both 8 hr (Juma et al., Br J Clin Pharmacol. 10:327-335 (1980); Juma et al., Eur J Clin Pharmacol. 19:443-451 (1981); Sladek, Pharmacol Ther. 37:301-355 (1988)).

To more closely mimic this latter, more common, CPA treatment schedule, 9L/P450 cells were treated with CPA for 8 hr and then assayed for their ability to survive and to metabolize CPA to 4-OH-CPA. Twenty-four hr after an 8 hr exposure to CPA, the p35-deficient 9L/P450 cells retained 97% of their initial CPA 4-hydroxylase activity. By 72 hr, however, the CPA metabolic activity of the p35-deficient cells was reduced to only ˜30% of the initial activity level (FIG. 3B, left panel). By contrast, 9L/P450/p35 cells displayed an increase in P450 activity over the course of the experiment, as indicated by the ˜6-fold higher CPA 4-hydroxylase activity when compared to 9L/P450 cells at the 72 hr time point. This differential effect of 8 hr CPA treatment on 9L/P450 vs. 9L/P450/p35 cells reflects two factors: 1) a decrease in the survival of 9L/P450 cells but not 9L/P450/p35 cells (FIG. 3B, middle panel); and 2) an even more rapid, and more complete, loss of CPA 4-hydroxylase activity in 9L/P450 cells (FIG. 3B, left panel).

Overall, the p35-expressing cells exhibited ˜2-3-fold higher CPA 4-hydroxylase specific activity (calculated by normalizing 4-OH-CPA production to total cell protein (i.e., crystal violet staining; A595 nm) beginning 24 hr after the initial 8 hr drug treatment period (FIG. 3B, right panel). Of note, 9L/P450 and 9L/P450/p35 cells displayed the same CPA 4-hydroxylase specific activity prior to CPA treatment (0-4 hr CPA samples).

Individual 9L/P450/p35 clones express differing amount of p35 and P450 reductase but similar amounts of P450. To further investigate the effects of p35 on P450-catalyzed CPA activation, four p35-expressing clones derived from the 9L/P450 cell line were isolated. The clones were characterized by RT-PCR as having a level of p35 expression that was either low (clones 3 and 7; designated p35-3 and p35-7) or high (clones 8 and 9; p35-8 and p35-9) (FIG. 4A). All four clones showed substantial caspase 3 inhibition (FIG. 4B). Western blot analysis of the individual clones showed no major differences in P450 2B6 expression, except for the p35-9 cells, where the expression was lower (FIG. 4C).

By contrast, the P450 reductase activity of the individual clones was variable panel D). These latter findings reflect the way the original 9L/P450 cell line was generated (Jounaidi et al., Cancer Res. 58:4391-4401 (1998); Jounaidi and Waxman, Cancer Research. 61:4437-4444 (2001)): a single P450 2B6-expressing clone of 9L cells was originally transduced with a P450 reductase-expressing retrovirus to generate a pool of 9L/P450 cells, comprised of individual cells with variable P450 reductase levels. Subsequent transduction of this pool with retrovirus encoding p35 gave rise to the variability in both p35 and P450 reductase expression seen in the individual clones in FIGS. 4A-4C.

9L/P450 cells that express high levels of p35 maintain their ability to activate CPA. FIG. 5 depicts cellular growth rates and CPA 4-hydroxylase activities of 9L/P450 cells and the four 9L/P450/p35 clones. Cells were untreated (FIG. 5A, across) or were treated with CPA either continuously (FIG. 5B) or for a single 8 hr exposure (FIG. 5C). All five cell lines exhibited similar growth rates in the absence of drug treatment (FIG. 5A, center panel), however, the intrinsic ability of each line to activate CPA varied. 9L/P450 cells and the low p35-expressing clone p35-3 displayed essentially the same rate of 4-OH-CPA generation at each of the four time points examined (i.e., 0-72 hr), while clone p35-7 displayed an increased rate of active metabolite generation (seen at t=0 and at t=24 hr), which may be explained by its elevated P450 reductase activity (c.f., FIG. 4D). Of the two high p35-expressing clones, p35-8 displayed a substantially increased rate of 4-OH-CPA production at all time points (c.f., elevated P450 reductase activity of this clone; FIG. 4D), while p35-9 showed a somewhat reduced rate of CPA metabolite formation, consistent with its lower P450 2B6 protein level (FIG. 4C).

Under conditions of continuous CPA treatment (FIG. 5B), only clone p35-9 showed a significant increase in cell survival and prolonged formation of 4-OH-CPA (48 and 72 hr; left panel). However, in cells treated with CPA for 8 hr, both clonal lines that express high levels of p35 showed a significant increase in their ability to generate activated CPA metabolite, with a very substantial 6.8-7.8 fold elevation of CPA 4-hydroxylase activity observed at the 72 hr time point compared to the p35-deficient controls (FIG. 5C, left panel). This elevated CPA 4-hydroxylase activity in part reflects maintenance by p35 of the cell's P450 specific activity, and contrasts with the CPA-induced loss of P450 specific activity in the absence of p35 (FIG. 5C, right panel). The ability of the 9L/P450/p35-8 cells to survive and to maintain high CPA 4-hydroxylase activity for prolonged time is of particular interest, insofar as these cells generate (and are exposed to) ˜2.5-fold higher amounts of the cytotoxic metabolite 4-OH-CPA during the 8 hr drug exposure period (FIG. 5C, left panel).

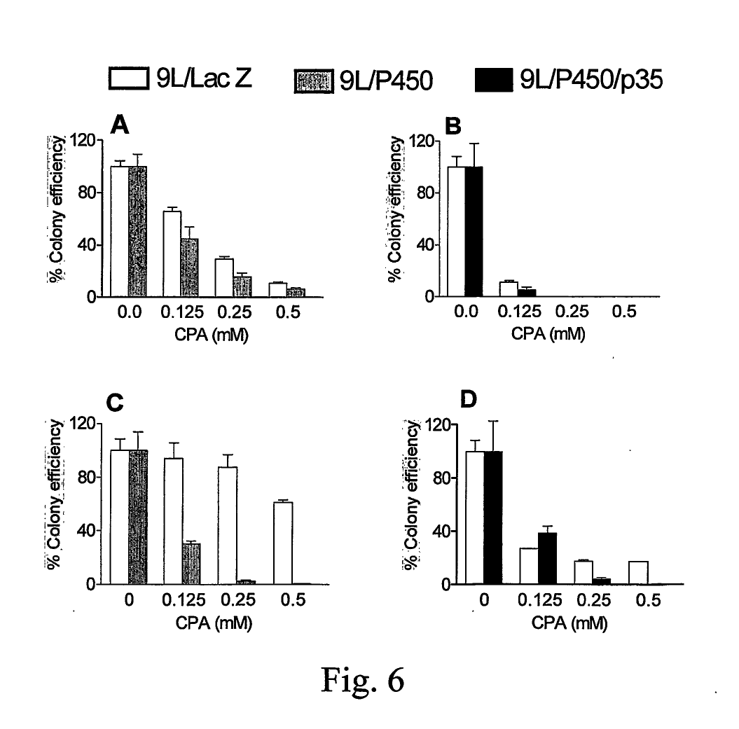

Co-expression of p35 enhances the P450-mediated bystander effect—We next investigated whether the enhanced CPA 4-hydroxylase activity exhibited by the 9L/P450/p35-9 cells at the time of the second 8 hr exposure to CPA would result in an increase in bystander killing by these cells. While 9L/P450 and 9L/P450/p35-9 cells were both able to confer killing on 9L/LacZ bystander cells, as determined in a colony formation assay (FIGS. 6A-6D), the extent of bystander killing in response to a second CPA treatment was much greater in the case 9L/P450/p35-9 cells, as seen in a mixed culture experiment (FIG. 6B vs. 6A). Moreover, when the bystander cells were separated from the 9L/P450 cells by a permeable membrane, CPA-pretreated 9L/P450 cells conferred little or no bystander cell killing in response to a second CPA treatment, whereas 9L/P450/p35-9 cells were able to confer 70-80% bystander cell killing under the same conditions (FIG. 6D vs. 6C).

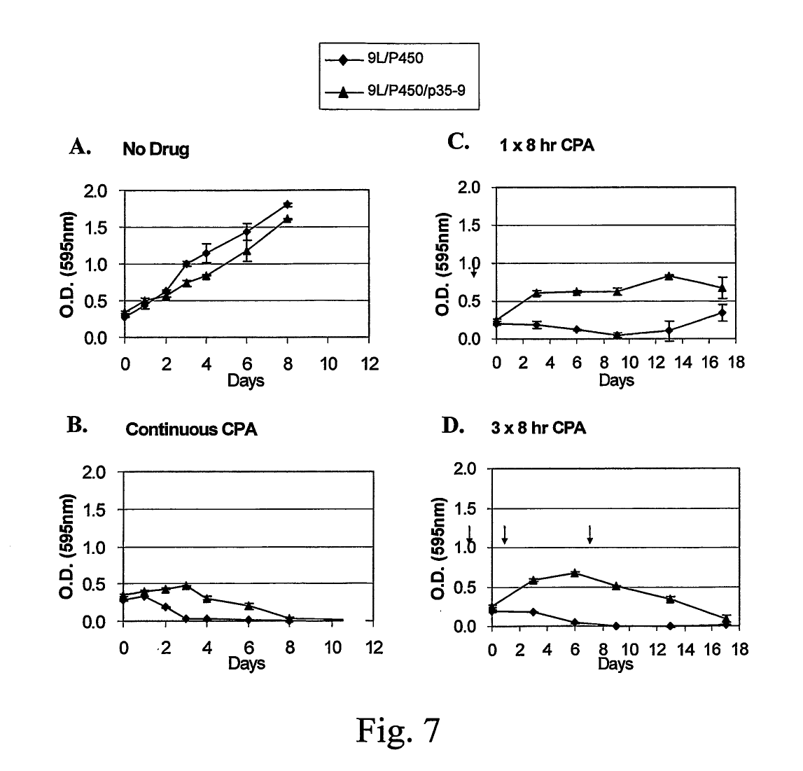

p35 expression does not block CPA-induced tumor cell death—We next evaluated the impact of high levels of p35 expression (e.g., in p35-9 cells) on the ultimate fate of CPA-treated 9L/P450 cells. FIGS. 7A-7D show that while p35 may delay tumor cell death, 9L/P450 tumor cells expressing p35 are eventually killed by CPA. 9L/P450 cells treated with CPA continuously died within 3 to 4 days, whereas an 8 day period was required for the death of 9L/P450/p35-9 cells (FIG. 7B). By contrast, a single 8 hr CPA treatment was not sufficient to kill 9L/P450 or 9L/P450/p35-9 cells. In the case of 9L/P450 cells, a substantial decrease in cell number was seen after 9 days, but this was followed by regrowth of individual colonies by 13-17 days (FIG. 7C). However, when the cells were given three 8 hr CPA treatments (vertical arrows, FIG. 7D), all of the 9L/P450 cells died by day 6-9 and the 9L/P450/p35-9 cells by day 17. These growth inhibition data reflect a true loss of tumor cell viability, as demonstrated in a colony formation assay (Table 1). 9L/P450 and 9L/P450/p35-9 cells were treated with CPA for 8 hr, on day 0 and again on day 3, and then incubated in drug-free media until day 6, at which time the cells were replated and colony formation quantitated 12 days later. 9L/P450 and 9L/P450/p35-9 cells both displayed similar colony formation activity in the absence of CPA treatment (Table 1). Furthermore, 9L/P450/p35-9 cells showed 2-fold higher colony formation activity compared to 9L/P450 cells after a single 8 hr CPA treatment (p<0.02). This difference reflects the lower initial exposure to 4-OH-CPA of the 9L/P450/p35-9 cells used in this experiment (c.f., lower intrinsic CPA 4-hydroxylase activity of the p35-9 clone; c.f, FIG. 5). However, when two 8 hr CPA treatments, spaced 3 days apart, were applied to the cells, no viable colonies were formed by either cell line. Thus, even in 9L/P450/p35-9 cells, which express the highest level of p35 and a reduced level of P450, two 8 hr CPA treatments are sufficient to effect complete cell killing, in agreement with the loss of colony formation activity seen for p35 cells in FIG. 6.

Table 1 shows the effect of CPA treatment on colony formation activity of 9L/P450 and 9L/P450/p35-9 cells. Cells plated in 12 well plates at 1.5×10⁵ cells/well/1.5 ml culture media were untreated or were treated with 1 mM CPA for a single 8 hr CPA treatment (=day 0) or for two 8 hr CPA treatment, on day 0 and day 3, respectively. Six days after the initial CPA treatment the cells remaining on each plate were counted and replated in duplicate as described in Materials and Methods. Eleven days after replating, the number of colonies (≧50 cells each) that had formed under each treatment condition was counted. Data shown represent mean±SD for n=3 and are based on cells plated at densities of 100 (untreated) or 1000, 5000 or 10,000 cells per well of a 6 well dish (CPA-treated samples). Where indicated (**), colony formation activity was significantly different between 9L/P450 and 9L/P450/p35-9 cells, as determined by two tailed, two sample equal variance t-test (p<0.02). Relative colony formation activities are shown in parenthesis.

TABLE 1

| | Colonies per 10,000 cells | |
|---|---|---|
| Treatment | 9L/P450 | 9L/P450/p35-9 |
| No Drug | 6950 ± 150 (100 ± 2.1) | 5250 ± 250 (100 ± 4.7) |
| 1 × 8 hr CPA | 77 ± (1.1 ± 0.5) | 150 ± 49 (2.9 ± 0.9)** |
| 2 × 8 hr CPA | 0 ± 0 | 0 ± 0 |

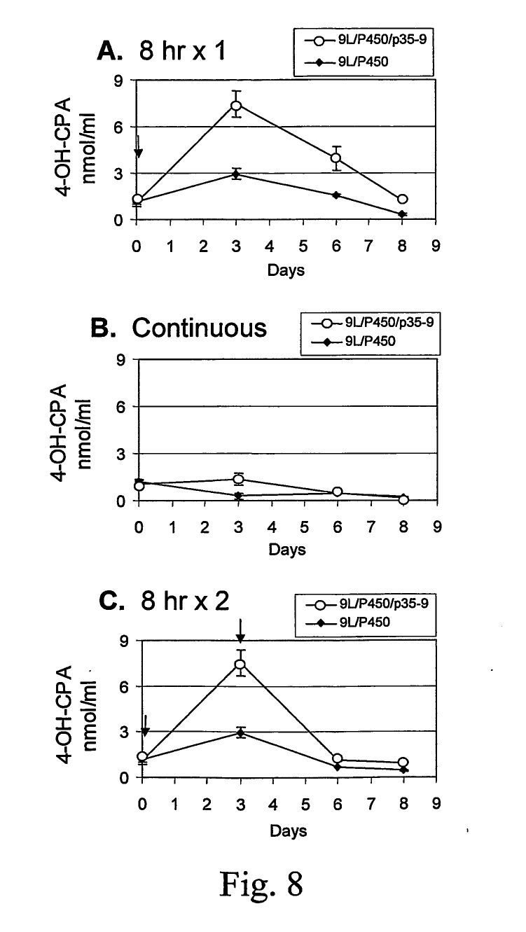

Analysis of the CPA 4-hydroxylase activity of the 9L/P450 and 9L/P450/p35-9 cultures revealed comparable levels of 4-OH-CPA production during the initial 8 hr drug treatment (FIGS. 8A-8D; data points plotted on y-axis) and only a modest difference between the two cell lines when exposed continuously to CPA (FIG. 8B; c.f., 3 day data points). By contrast, a substantial difference in cellular CPA 4-hydroxylase activity was seen at the time of the second CPA treatment (FIG. 8C). However, by day 6, when a putative third CPA treatment could be given, the 9L/P450/p35-9 cells no longer retained their ability to activate CPA (FIG. 8C).

We have identified ways to optimize the capacity of tumor cells that express prodrug activating genes to kill bystander tumor cells that are not transduced with the prodrug-activating gene. An important feature of GDEPT strategies, including P450 GDEPT, is the bystander effect, which can extend the cytotoxic effect of the prodrug to include tumor cells that are proximal to tumor cells transduced with the therapeutic gene (Chen and Waxman, Current Pharmaceutical Design. 8:99-110 (2002); Springer and Niculescu-Duvaz, J Clin Invest. 105:1161-1167. (2000)). Activated CPA metabolites produced by P450-expressing tumor cells kill P450-deficient tumor cells in a cell contact-independent manner. However, the P450-expressing tumor cells are somewhat more sensitive to activated drug than the P450-deficient tumor cells (Chen and Waxman, Cancer Res. 55:581-589 (1995)). Depletion of cellular GSH by BSO treatment was shown to increase the tumor cell's sensitivity to activated CPA, as anticipated; however this treatment also decreased the cell's potential to generate activated CPA metabolites (FIG. 1). BSO treatment thus does not serve as a useful way to enhance P450 GDEPT, insofar as the accompanying decrease in production and release of cytotoxic CPA metabolites into the culture media decreases the cell's ability to produce cytotoxic, bystander metabolites.

We also discovered that P450-transduced tumor cells lose functional P450 activity early during the course of CPA-induced cell death, thereby shortening the time frame during which these cells generate activated CPA metabolites and release them into the culture medium. When CPA treatment was limited to an 8 hr exposure period, the cells died more slowly, however their ability to continue to activate CPA did not improve greatly. By contrast, when the anti-apoptotic factor p35 was coexpressed with P450, the potential of the P450-transduced tumor cells for continued production of active CPA metabolites was greatly enhanced.

Retroviral expression of p35 in combination with the CPA-activating P450 2B6 not only prolonged tumor cell longevity following an 8 hr CPA treatment, but also provided for long-term maintenance of the tumor cell's capacity for active metabolite generation. As a results, p35 co-expression led to a substantial increase in P450-mediated bystander killing. The rate of 4-OH-CPA production by 9L/P450/p35 cells was found to be up to about 5-fold higher, on a per cell protein basis, than in 9L/P450 cells given an 8 hr exposure to CPA. The prolonged survival, in combination with the enhanced production of active CPA metabolites resulted in an about 7-8-fold overall increase in the prodrug actication capacity of p35-co-staining cells 3 days after the initial CPA treatment. This finding was observed in a pool of p35-transduced cells, and was confirmed in two independent p35-expressing clonal cell lines (FIG. 5).

When p35-expressing 9L tumor cells were exposed to CPA in a continuous manner, although there was little or no increase in the CPA 4-hydroxylase activity of the p35-expressing cells when normalized on a per mg cell protein basis, p35 did still confer the advantages associated with prolonged cell survival and the accompanying increase in active metabolites generated by the larger number of tumor cells maintained on the plates.

Several different protocols for CPA administration are currently in clinical use. CPA can be administered either at a single high dose or as fractionated doses given over a short period of time, based on the concept of maximum tolerable dose. Subsequent CPA treatments are administered 2-4 wk later, at a time when the patient has recovered from systemic toxicity associated with the initial chemotherapy. In a second schedule, smaller doses of CPA are given daily over a longer period of time (Dorr and Van Hoff, 319-328 (1994)).

A third treatment regimen, which has been used in patients with advanced malignancies and is made possible by the use of autologous bone marrow transplantation, involves treatment with high dose CPA, typically administered to patients continuously as an infusion given over a 4 day period (Ayash et al., J Clin Oncol. 10:995-1000 (1992)). In the present study, optimal production of active CPA metabolites was achieved in tumor cells co-expressing P450 and p35 using a schedule of sequential CPA treatments at moderate doses, spaced by short recovery interval lasting only a few days. The use of a repeated CPA dosing schedule, in place of a single bolus exposure or continuous drug exposure, is supported by mouse model studies, where CPA administered at a moderate dose using a schedule of drug treatment repeated every 6 days (in the absence of P450 GDEPT) was shown to have an anti-angiogenic component, and was far more effective at inducing tumor cell apoptosis, yielding a higher cure rate than the conventional maximum tolerable dose approach (Browder et al., Cancer Res. 60:1878-1886 (2000)). These findings were confirmed and extended in in vivo tumor model studies where the 6 day repeat CPA schedule resulted in complete or near complete regression of large tumors that express P450 2B6 in combination with P450 reductase (Jounaidi and Waxman, Cancer Research. 61:4437-4444 (2001)).

Our discovery of the advantage of delivering to tumor cells an anti-apoptotic factor, such as p35, in the context of a prodrug activation gene therapy was surprising, in view of the numerous studies that report the potential therapeutic utility of pro-apoptotic factors in cancer treatment (Opalka B et al (2002) Cells Tissues Organs 172, 126-132). Therefore, the introduction of anti-apoptotic factors into human cells to advance cancer treatment is counter-intuitive. Moreover, this approach may seem, at first glance, to be potentially dangerous, in view of the many reports that describe anti-apoptotic factors, such as Bcl-2 and Survivin, and their role in tumorigenesis and chemoresistance (Deveraux and Reed, Genes Dev. 13:239-252 (1999); Reed, Apoptosis and Cancer Chemotherapy 99-116 (1999); Zornig et al., Biochim Biophys Acta. 1551:F1-37 (2001)). However, therapeutic expression of anti-apoptotic factors, such as p35, has been suggested for the treatment of neurodegenerative diseases such as Alzheimer's or Parkinson's in order to maintain neuronal viability (Aebischer and Ridet, Trends Neurosci. 24:533-540 (2001); Deigner et al., Expert Opin Investig Drugs. 9:747-764 (2000)) and major safety concerns have not emerged from such studies.

Moreover, the success of the novel approach exemplified in this study, in which expression of an anti-apoptotic gene is coupled to a suicide gene therapy, is based upon the fact that p35 and other anti-apoptotic factors prolong but ultimately do not block tumor cell death. Even in tumor cells where prodrug activation activity was sub-maximal and where p35 was expressed at a high level, two 8 hr prodrug treatments spaced 3 days apart were found to be sufficient to effect tumor cell death, as exemplified for the clonal tumor cell line 9L/P450/p35-9. Importantly, p35 expression did not increase tumor cell survival in long-term clonogenic assays, evidencing that the damage induced by treatment with the anti-cancer drug is sufficient to kill tumor cells, despite the expression of a strong caspase inhibitor (Table 1).

Given the imprecise nature of gene delivery, the possibility exists that the anti-apoptotic factor might be transduced into and subsequently expressed in individual tumor cells in the absence of the suicide gene. This occurrence may be circumvented in several ways, including coexpression of the suicide gene and p35 as a single transcript using an internal ribosome entry site (IRES), or by placing the p35 gene under the control of a regulatable promoter. Furthermore, the anticipated immunogenicity of p35, a baculovirus-encoded protein, may help to ensure that all of the p35-expressing tumor cells are ultimately eliminated by the host's immune system.

Alternatively, p35 may be incorporated into a cellular vector for gene delivery, such that the anti-apoptotic gene is never introduced into the tumor itself. For example, one GDEPT approach, which has progressed through phase I and phase II clinical trials, uses an encapsulated cellular vector that is engineered to express P450 and can be used to deliver prodrug activation activity directly to pancreatic tumors (Lohr et al., Lancet. 357:1591-1592 (2001); Muller et al., Ann N Y Acad Sci. 880:337-351 (1999)). The use of encapsulated cells that have been genetically modified to express both a prodrug activation gene and a caspase inhibitor such as p35 would allow for the regulated expression of both genes in the cell population, with the additional advantage that no direct gene therapeutic intervention in the target tumor cell is necessary. Encapsulated cells offer the added advantage of circumventing immune responses, which can be significant in the case of viral vectors, such as adenoviral vectors (Lohr et al., Lancet. 357:1591-1592 (2001)).

The present finding that P450-expressing tumor cells treated with CPA ultimately die, even in the presence of high levels of the potent pan-caspase inhibitor p35, demonstrates that caspase activation is not a prerequisite for CPA-induced cell death. The fact that the anti-apoptotic factor Bcl-2 prolongs survival of tumor cells exposed to activated CPA (Schwartz and Waxman, Mol Pharmacol. 60:1268-1279. (2001)) evidences that mitochondria play a pivotal role in regulating CPA-induced cell death. The present finding that caspase inhibition does not increase the long-term clonogenic survival of CPA-treated 9L/P450 tumor cells further suggests that CPA-induced mitochondial transition, which is responsible for the release of multiple proapoptotic factors, including caspases (Susin et al., J Exp Med. 189:381-394 (1999)), cytochrome C (Green and Reed, Science. 281:1309-1312 (1998); Slee et al., J Cell Biol. 144:281-292 (1999)), apoptosis-inducing factor (AIF) (Joza et al., Nature. 410:549-554 (2001); Susin et al., Nature. 397:441-446 (1999)) and Smac/Diablo (Du et al., Cell. 102:33-42 (2000); Verhagen et al., Cell. 102:43-53 (2000)), occurs upstream of the activation of caspases. This finding confirms that CPA induces a mitochondrial caspase 9-dependent pathway of cell death that can be inhibited by Bcl-2 (Schwartz and Waxman, Mol Pharmacol. 60:1268-1279 (2001)).

The pro-apoptotic protein Smac/Diablo, which is released to the cytosol following mitochondrial transition, is of particular interest in that its primary function it to bind to and neutralize the inhibitory activities of several IAPs, including hIAP1, hIAP2, XIAP and Survivin (Du et al., Cell. 102:33-42 (2000); Verhagen et al., Cell. 102:43-53 (2000)). The possibility of Smac/Diablo release in tumor cells exposed to 4-OH-CPA, in combination with the intrinsic high inhibitory constant of the human IAPs, hIAP1, hIAP2 and Survivin, in comparison to that of p35 (Deveraux and Reed, Genes Dev. 13:239-252 (1999)), may contribute to the less effective caspase inhibition that is presently described for 9L/P450 cell lines that stably expressed these factors. Additionally, the release of Smac/Diablo could explain the need for relatively high levels of p35 expression in order to prolong tumor cell death. The apoptosis-inducing factor AIF is another factor released from mitochondria and may contribute to CPA-induced cell death in the presence of the caspase inhibitor p35 (Susin et al., Nature. 397:441-446 (1999)) (Joza et al., Nature. 410:549-554 (2001)), insofar as AIF has been shown to lead to caspase-independent programmed cell death. AIF is sequestered in the mitochondrial intermembrane space, and when released following apoptotic stimulation, translocates to the nucleus where it causes large-scale chromatin fragmentation (Susin et al., Nature. 397:441-446 (1999)). These effects are not blocked by the general caspase inhibitor z-VAD.fink, although, the release of AIF from mitochondria is regulated by Bcl-2 (Susin et al., J Exp Med. 189:381-394 (1999)).

Baculoviruses have evolved the ability to express anti-apoptotic factors such as p35 and p49, enabling these insect cell viruses to suppress the host cell death response to viral infection and thereby maximize virus production. The concept of using anti-apoptotic factors to enhance GDEPT and other gene therapies for cancer treatment is exemplified by the present study and is expected to be directly applicable to prodrug activation gene therapy using other clinically established P450 prodrugs, at least 10-12 of which are known (Chen and Waxman, Current Pharmaceutical Design. 8:99-110 (2002)).

Additional anti-apoptotic approaches, including expression of Bcl-2 family members with anti-apoptotic activity, mammalian IAPs, antisense or siRNA targeting of caspases, or expression of factors that regulate receptor-mediated cell death, such as the Fas inhibitor FLIP or the Trail decoy receptors, are viable alternative approaches to couple with prodrug activation or other gene therapies that benefit from an enhanced bystander effect. Gene therapies that would be improved in this manner include other established prodrug activation therapies, such as those that use Herpes Simplex Virus thymidine kinase, cytosine deaminase or other known suicide genes as well as therapies based on the expression of soluble or secretable factors that have anti-angiogenic, cytotoxic or immune modulatory activity.

It is expected that these gene therapy combinations and their bystander cytotoxic effects will be enhanced by combination with p35 or other anti-apoptotic factors, although the extent of this enhancement may be dependent upon the cell death mechanism induced by the activated prodrug or therapeutic factor, the extent of cell death inhibition conferred by the anti-apoptotic factor chosen, and the extent of associated increase in bystander killing conferred by expression of the anti-apoptotic factor. The ability of IAPs to prolong prodrug-induced cell death in a manner that allows for enhanced production of activated drug metabolites without increasing tumor cell survival can thus provide a general way to enhance a broad range of gene-based and cell therapy-based cancer treatments.

Our findings demonstrate that the IAP and caspase inhibitor p35 is able to substantially delay, but not block, prodrug cytotoxicity to tumor cells expressing a prodrug-activation enzyme, thereby providing for the prolonged production of active drug metabolite and its release into the tumor milieu, with a corresponding increase in bystander cytotoxicity. The implications of these findings are discussed in the context of the proposed incorporation of IAPs and other anti-apoptotic factors into gene therapies that benefit from a bystander cytotoxic effect.

Impact of Caspase Inhibitor p35 on P450 Bystander Activity. As noted, we have observed that tumor cell P450 activity declines appreciably during the course of apoptosis induced by CPA, limiting the tumor cell's potential for continued production of activated drug metabolites. To overcome these limitations, we investigated whether anti-apoptotic factors could be used to enhance CPA's bystander activity. Detailed studies were performed with one anti-apoptotic factor, the pan-caspase inhibitor p35 (Clem R J. (2001) Cell Death Differ 8: 137-143). Retroviral expression of p35 substantially prolonged the activation of CPA by P450-expressing 9L tumor cells, resulting in a strong increase in bystander cytotoxicty toward P450-deficient tumor cells. p35 did not, however, affect tumor cell growth kinetics or induce drug resistance, as shown by the absence of long-term tumor cell survival or detectable colony formation activity following CPA treatment. Thus, caspase activation is not a prerequisite for CPA-induced tumor cell death. These findings provide proof-of-principle for the use of anti-apoptotic factors, such as p35, to enhance prodrug activation gene therapy by delaying tumor cell death, thereby increasing the net production of bystander cytotoxic metabolites and hence the overall effectiveness of the anti-cancer strategy.

Regulated expression of p35. A key feature of p35 is that it is a broad-spectrum caspase inhibitor that blocks CPA-induced apoptosis downstream of the critical mitochondrial transition step. Mitochondrial transition accompanied by the release of cytochrome C and apoptosis-inducing factor (Joza N. et al. (2001) Nature 410: 549-554) is a key event that commits the cell to death; it leads to collapse of the electron transport chain (Green D R & Reed J C. (1998) Science 281: 1309-1312) and the irreversible loss of clonogenic activity via a necrotic process (Brunet C L., et al. (1998) Cell Death Differ 5: 107-115; Amarante-Mendes G P., et al. (1998) Cell Death Differ 5: 298-306; Brown J M & Wouters B G (1999) Cancer Res 59: 1391-1399; Xue L, et al. (2001) Curr Biol 11: 361-365). Importantly, the downstream activation of caspases is not required for cell death; rather, it serves to accelerate and guide cell death along an orderly, apoptotic pathway (Green D R & Reed J C., Id.). Consequently, p35 can be incorporated into a P450-based gene therapy in a manner that will not only be effective but is safe. To provide additional measures of safety, the expression of p35 can be regulated using a repressible promoter system (‘Tet-off’ system) and in a manner that is closely linked to the expression of P450.

The possibility of long-term survival of a tumor cell population transduced with p35 can be greatly diminished by placing the p35 gene under the control of a strong, repressible promoter, such as the ‘Tet-off’ expression system (Clontech, Inc.). The Tet-off system is ideally suited for achieving maximal control of transgene expression, which can be suppressed in a wide range of cells both in vitro and in vivo (Mayford M, et al. (1996) Science 274: 1678-1683; Bohl D, et al. (1997) Nat Med 3: 299-305). Tetracycline is a well-tolerated antibiotic, and near complete suppression of gene expression can be achieved at doses that are very low (Mizuguchi H & Hayakawa T, (2002) J Gene Med 4: 240-247) and have no cytotoxicity or other significant effects on cell proliferation or animal growth, even with continuous treatment (Mayford M, et al. (1996) Science 274: 1678-1683; Bohl D, et al. (1997) Nat Med 3: 299-305). Adenoviral vectors that incorporate a regulated transgene in the viral E1 deletion region and the tetracycline-responsive transcriptional activator gene in the E3 deletion region have been described Mzuguchi H & Hayakawa T, Id.) and can be used to deliver the p35 cDNA (1 kb) in combination with a minimal CMV-P450 expression cassette (˜2.6 kb) or a CMV-P450-IRES-P450R expression cassette (˜5.3 kb). Use of such a vector provides for tetracycline suppression of p35 expression, thereby enabling the CPA- and P450-dependent killing of any residual tumor cells that may have initially been protected by p35 from 4-OH-CPA cytotoxicity. This Tet-off adenovirus provides for very tight regulation, with 20- to 500-fold suppression of transgene expression achievable by treatment with very low tetracycline doses (Mizuguchi H & Hayakawa T, Id.). Any such low level, leaky expression of p35 would not reach the threshold required to block CPA-induced cell death.

In vivo delivery of p35—Tumor-selective gene delivery can readily be accomplished using a replication-deficient adenoviral vector that expresses both P450 and p35, administered in combination with a replicating helper virus such as the E1b region 55 kd-deleted Onyx adenovirus to enhance tumor cell-selective viral spread. The replication-defective virus Adeno-P450/p35 can be constructed using the unique MfeI site of the Adeno-X shuttle plasmid pShuttle-P450 2B6-IRES-P450R to incorporate a short CMV-p35 expression cassette (˜2.1 kb). The overall 7.6 kb size of the resultant pShuttle-derived fragment includes separate expression cassettes encoding p35 and P450-IRES-P450R and is within the 8 kb size that can readily be incorporated into pAdeno-X. The shuttle plasmid's unique 7.6 kb I-CeuI-III-Σ_{χ}εI fragment can be ligated into pAdeno-X, after which the resulting adenoviral DNA can be linearized, transfected into 293 cells and the replication-defective recombinant virus Adeno-P450/p35 then harvested using established methods. The resulting Adeno-P450/p35 virus can be used to effect the coordinated expression of P450 and p35. Furthermore, Adeno-P450/p35 can be co-administered in combination with a tumor cell-replicating virus such as the Onyx adenovirus to achieve tumor-selective gene delivery. The tumor cell-replicating virus can be administered in combination with Adeno-P450/p35 at a ratio determined empirically to enhance the tumor cell spread of the latter virus determined by analysis using cryosectioning and immunostaining with anti-P450 2B6 monoclonal antibody. The impact of p35 co-expression with P450 can be monitored by double immunofluorescence analysis of cryosectioned tumors using TUNEL staining (apoptosis-associated DNA fragmentation) (Schwartz P S & Waxman D J, (2001) Mol Pharmacol 60: 1268-1279) in combination with anti-P450 2B6 immunofluorescence, applied to tumors excised after CPA treatment, to monitor bystander killing of the Adeno-P450/p35-treated tumors. p35 co-expression will block apoptosis and therefore decrease TUNEL staining of the P450-stained tumor cells, but will enhance the net production of 4-OH-CPA and thereby increase apoptosis and TUNEL staining of the surrounding, P450-deficient bystander cells.

Impact of p35 on adenovirus transmission—Adenoviruses encode several genes that subvert host defense mechanisms and facilitate replication and transmission of the virus to surrounding cells. For example, the adenoviral E1b-19k protein, a functional homolog of the mitochondrial anti-apoptotic factor Bcl-2, suppresses host cell apoptosis to insure that the cell does not die prematurely, i.e., prior to completion of adenoviral replication (Cuconati A & White E. (2002) Genes Dev 16: 2465-2478). This anti-apoptotic effect, and the associated production of viral progeny, is likely to be augmented in cells that express p35, as revealed by the enhancing effect that p35 has on adenoviral spread using the tumor cell-replicating virus helper system. The proposed benefit of delaying host cell death is supported by the finding that deletion of the adenoviral E3b region ADP gene (adenoviral death protein) prolongs host cell survival and can increase the expression of an adenovirus-encoded CD enzyme, with a concomitant increase in the activation of 5-FC to 5-FU (Hawlins L K & Hermiston T W. (2001) Gene Ther 8: 1132-1141).

In experiments carried out in 9L cells that express p35 constitutively we have found a 3.5-fold increase in helper use-mediated transmission of Adeno-βgal compared to p35-deficient 9L controls (FIG. 9). Thus, we have discovered that by introduction of an anti-apoptotic factor that is designed to enhance bystander activity, we can achieve the added benefit of improved viral transmission of the therapeutic gene within a population of tumor cells. This effect is expected to be achieved with other anti-apoptotic factors, as well as with other replicating viral vectors, which also will benefit from the prolonged death of cells infected by the replicating virus, insofar as this will lead to a net overall increase in production of viral progeny prior to host cell death. p35 could, alternatively, be incorporated directly into the replicating viral vector, however, it is preferable to express p35 from the defective virus, to minimize the possibility that p35 will be introduced into tumor cells without the prodrug activation or other therapeutic P450 drug susceptibility gene. p35 is also less likely to interfere with the early stages of virus amplification and release when its expression is limited to tumor cells that are co-infected with the replication defective virus in combination with the replicating virus, which we have found initially correspond to a comparatively minor fraction of the overall tumor cell population.

p35 and systemic anti-tumor immune response. The in vivo bystander cytotoxicity associated with tumor cell-activated prodrugs, such as HSV-tk-activated GCV and CD-activated 5-FC, can be comprised of three distinct components: 1) a localized tumor cell bystander effect that results from diffusion (or gap junctional transfer) of the active metabolite to bystander cells; 2) an endothelial cell bystander effect; and 3) a systemic bystander effect that reflects a cell-mediated immune response that targets the drug-treated tumor cells. This latter effect is readily identified by the much stronger GDEPT bystander response that occurs when prodrug is used to treat syngeneic tumors grown in an immunocompetent host as compared to an immunodeficient host (i.e., nude or scid mice)(Pope I M, et al. (1997) Eur J Cancer 33: 1005-1016; Kuriyama S, et al. (1999) Int J Cancer 81: 592-597). A systemic bystander effect is of great therapeutic value in treating disseminated metastatic disease. Systemic bystander activity is more readily induced when tumor cells undergo necrotic death as compared to death by apoptosis, as is expected to be the case for tumor cells that die under conditions where apoptosis is inhibited, such as in the case of CPA-treated tumor cells that co-express P450 and p35. Evidence for such a systemic bystander effect was obtained in a P450 GDEPT clinical trial, where regression of distal, untreated surface breast tumor nodules was associated with an apparent anti-tumor immune response following localized delivery of CYP2B6 and CPA treatment (Kingsman S. (2002) Amer Assoc Pharmaceut Sci presentation, Nov. 12, 2002, Toronto, Canada; and http://www.oxfordbiomedica.co.uk/metxia.htm.). It is expected that foreign anti-apoptotic factors such as p35 will enhance this systemic bystander effect by virtue of their intrinsic antigenicity, and by the shift in drug-induced cell death from an apoptotic mechanism, in the absence of p35, to a necrotic pathway of cell death in its presence Schwartz P S & Waxman D J. (2001) Mol Pharmacol 60: 1268-1279).

p35 and bystander killing effect of prodrug activation systems based on other enzymes, such as cytosine deaminase (CD) and herpes simplex virus thymidine kinase (HSV-tk). The concept that underlies the increase in P450/CPA bystander activity seen in tumor cells transduced with p35 is expected to be broadly applicable to other prodrug activation systems that activate caspase-dependent apoptotic mechanisms (Schwartz P S, et al. (2002) Cancer Res 62: 6928-6937). These include the well-studied enzyme/prodrug combinations, CD/5-FC and HSV-tk/GCV, both of which activate caspase-dependent apoptotic pathways that may be subject to inhibition by p35. In the case of 5-FU, the active metabolite of 5-FC, apoptosis is initiated by caspase 8 (Adachi Y, et al. (1999) Int J Oncol 15: 1191-1196; Aota et al., (2000) Biochem Biophys Res Commun 273: 1168-1174; Ohtani et al. (2000) Anticancer Res 20: 3117-3121). In contrast, GCV triphosphate, the active metabolite of GCV, induces caspase 8-mediated cell death in tumor cells that have a functional p53 (Beltinger C, et al. (1999) Proc Natl Acad Sci U S A 96: 8699-8704), whereas caspase 9-dependent cell death is induced in p53-deficient cells (Tomicic M F, et al. (2002) Oncogene 21: 2141-2153). A potentially important difference between these two prodrug systems is that 5-FU diffuses freely from cell to cell and exerts strong cell contact-independent bystander activity (Huber B E, et al. (1994) Proc Natl Acad Sci USA 91: 8302-8306), whereas the bystander activity of GCV requires direct cell-cell contact, with gap junctions playing an essential role in transporting the active, phosphorylated metabolites from cell to cell (Mesnil M & Yamasaki H. (2000) Cancer Res 60: 3989-3999). This latter bystander effect is relatively weak, and contributes to the limited effectiveness of HSV-tk-based gene therapies. However, even in the case of CD, transduced ‘5-FU factory’ tumor cells display up to a 500-fold greater susceptibility to killing than do bystander tumor cells (Lawrence T S, et al. (1998) Cancer Res 58: 2588-2593.). Consequently, the co-expression of p35 is expected to increase bystander activity in this case as well.

p35 is currently being considered for other therapeutic applications that may benefit from the inhibition of apoptosis, including the treatment of seizure-associated neurodegenerative disease and the increased cardiac cell death that is associated with ischemia/reperfusion, and thus far, safety problems have not emerged (Viswanath V, et al. (2000) Proc Natl Acad Sci USA 97: 2270-2275; Date T, et al. (2002) Cardiovasc Res 55: 309-319).

### REFERENCES

1. Aebischer P. and Ridet J. Recombinant proteins for neurodegenerative diseases: the delivery issue, Trends Neurosci. 24: 533-540, 2001.
2. Aghi, M., Hochberg, F. Breakefield, X. O. (2000) Prodrug activation enzymes in cancer gene therapy. J Gene Med 2, 148-164.
3. Ambrosini G., Adida C. and Altieri D. C. A novel anti-apoptosis gene, survivin, expressed in cancer and lymphoma, Nat Med. 3: 917-921, 1997.
4. Antman K., Ayash L., Elias A., Wheeler C., Hunt M., Eder J. P., Teicher B. A., Critchlow J., Bibbo J., Schnipper L. E. and et al. A phase II study of high-dose cyclophosphamide, thiotepa, and carboplatin with autologous marrow support in women with measurable advanced breast cancer responding to standard-dose therapy, J Clin Oncol. 10: 102-110, 1992.
5. Ashkenazi, A. and Dixit, V. M. (1999) Apoptosis control by death and decoy receptors. Curr Opin Cell Biol, 11, 255-260.
6. Ashkenazi A. and Dixit V. M. Death receptors: signaling and modulation, Science. 281: 1305-1308, 1998.
7. Ayash L. J., Wright J. E., Tretyakov O., Gonin R., Elias A., Wheeler C., Eder J. P., Rosowsky A., Antman K. and Frei E., 3rd Cyclophosphamide pharmacokinetics: correlation with cardiac toxicity and tumor response, J Clin Oncol. 10: 995-1000, 1992.
8. Baker, S. J. and Reddy, E. P. (1998) Modulation of life and death by the TNF receptor superfamily. Oncogene, 17, 3261-3270.
9. Browder T., Butterfield C. E., Kraling B. M., Shi B., Marshall B., O'Reilly M. S. and Folknan J. Antiangiogenic scheduling of chemotherapy improves efficacy against experimental drug-resistant cancer, Cancer Res. 60: 1878-1886, 2000.
10. Chen G. and Waxman D. J. Role of cellular glutathione and glutathione S-transferase in the expression of alkylating agent cytotoxicity in human breast cancer cells, Biochem Pharmacol. 47: 1079-1087, 1994.
11. Chen G. and Waxman D. J. Identification of glutathione S-transferase as a determinant of 4-hydroperoxycyclophosphamide resistance in human breast cancer cells, Biochem Pharmacol. 49: 1691-1701, 1995.
12. Chen L. and Waxman D. J. Intratumoral activation and enhanced chemotherapeutic effect of oxazaphosphorines following cytochrome P-450 gene transfer: development of a combined chemotherapy/cancer gene therapy strategy, Cancer Res. 55: 581-589, 1995.
13. Chen L. and Waxman D. J. Cytochrome P450 Gene-Directed Enzyme Prodrug Therapy (GDEPT) for Cancer, Current Pharmaceutical Design. 8: 99-110, 2002.
14. Chen L., Yu L. J. and Waxman D. J. Potentiation of cytochrome P450/cyclophosphamide-based cancer gene therapy by coexpression of the P450 reductase gene, Cancer Res. 57: 4830-4837, 1997.
15. Clem R. J., Fechheimer M. and Miller L. K. Prevention of apoptosis by a baculovirus gene during infection of insect cells, Science. 254: 1388-1390, 1991.
16. Deigner H. P., Haberkom U. and Kinscherf R. Apoptosis modulators in the therapy of neurodegenerative diseases, Expert Opin Investig Drugs. 9: 747-764, 2000.
17. Deveraux Q. L. and Reed J. C. IAP family proteins—suppressors of apoptosis, Genes Dev. 13: 239-252, 1999.
18. Dorr R., T., and Van Hoff D., D. Cancer Chemotherapy Handbook, 2nd edition, p. 319-328. Norwalk Conn.: Appleton & Lange, 1994.
19. Du C., Fang M., Li Y., Li L. and Wang X. Smac, a mitochondrial protein that promotes cytochrome c-dependent caspase activation by eliminating IAP inhibition, Cell. 102: 33-42, 2000.
20. Green D. R. Apoptotic pathways: the roads to ruin, Cell. 94: 695-698, 1998.
21. Green D. R. and Reed J. C. Mitochondria and apoptosis, Science. 281: 1309-1312, 1998.
22. Hecht J., Jounaidi Y. and Waxman D. J. Construction of P450-expressing Tumor Cell Lines Using Retroviruses. In: W. Walther and U. Stein (eds.), Gene Therapy of Cancer, pp. 85-94. Totowa, N.J.: Humana Press, 2000.
23. Huang Z. and Waxman D. J. High-performance liquid chromatographic-fluorescent method to determine chloroacetaldehyde, a neurotoxic metabolite of the anticancer drug ifosfamide, in plasma and in liver microsomal incubations, Anal Biochem. 273: 117-125, 1999.
24. Jounaidi Y., Hecht J. E. and Waxman D. J. Retroviral transfer of human cytochrome P450 genes for oxazaphosphorine-based cancer gene therapy, Cancer Res. 58: 4391-4401, 1998.
25. Jounaidi Y. and Waxman D. J. Frequent, Moderate Dose Cyclophosphamide Administration Improves the Efficacy of P450/P450 Reductase-based Cancer Gene Therapy, Cancer Research. 61: 4437-4444, 2001.
26. Joza N., Susin S. A., Daugas E., Stanford W. L., Cho S. K., Li C. Y., Sasaki T., Elia A. J., Cheng H. Y., Ravagnan L., Ferri K. F., Zamzami N. et al. Essential role of the mitochondrial apoptosis-inducing factor in programmed cell death, Nature. 410: 549-554, 2001.
27. Juma F. D., Rogers H. J. and Trounce J. R. The pharmacokinetics of cyclophosphamide, phosphoramide mustard and nor-nitrogen mustard studied by gas chromatography in patients receiving cyclophosphamide therapy, Br J Clin Pharmacol. 10: 327-335, 1980.
28. Juma F. D., Rogers H. J. and Trounce J. R. Effect of renal insufficiency on the pharmacokinetics of cyclophosphamide and some of its metabolites, Eur J Clin Pharmacol. 19:443-451, 1981.
29. Kagawa S., Gu J., Swisher S. G., Ji L., Roth J. A., Lai D., Stephens L. C. and Fang B. Antitumor effect of adenovirus-mediated Bax gene transfer on p53-sensitive and p53-resistant cancer lines, Cancer Res. 60: 1157-1161, 2000.
30. Kagawa S., He C., Gu J., Koch P., Rha S. J., Roth J. A., Curley S. A., Stephens L. C. and Fang B. Antitumor activity and bystander effects of the tumor necrosis factor-related apoptosis-inducing ligand (TRAIL) gene, Cancer Res. 61: 3330-3338, 2001.
31. Kan O., Griffiths L., Baban D., Iqball S., Uden M., Spearman H., Slingsby J., Price T., Esapa M., Kingsman S., Kingsman A., Slade A. et al. Direct retroviral delivery of human cytochrome P450 2B6 for gene-directed enzyme prodrug therapy of cancer, Cancer Gene Ther. 8: 473-482, 2001.
32. Komata T., Kondo Y., Kanzawa T., Hirohata S., Koga S., Sumiyoshi H., Srinivasula S. M., Bama B. P., Germano I. M., Takakura M., Inoue M., Alnenri E. S. et al. Treatment of malignant glioma cells with the transfer of constitutively active caspase-6 using the human telomerase catalytic subunit (human telomerase reverse transcriptase) gene promoter, Cancer Res. 61: 5796-5802, 2001.
33. Li P., Nijhawan D., Budihardjo I., Srinivasula S. M., Ahmad M., Alnenri E. S. and Wang X. Cytochrome c and dATP-dependent formation of Apaf-1/caspase-9 complex initiates an apoptotic protease cascade, Cell. 91: 479-489, 1997.
34. Lohr M., Hoffineyer A., Kroger J., Freund M., Hain J., Holle A., Karle P., Knofel W. T., Liebe S., Muller P., Nizze H., Renner M. et al. Microencapsulated cell-mediated treatment of inoperable pancreatic carcinoma, Lancet. 357: 1591-1592, 2001.
35. Micheau O., Solary E., Hammann A. and Dimanche-Boitrel M. T. Fas ligand-independent, FADD-mediated activation of the Fas death pathway by anticancer drugs, J Biol. Chem. 274: 7987-7992, 1999.
36. Moore M. J. Clinical pharmacokinetics of cyclophosphamide, Clin Pharmacokinet. 20: 194-208, 1991.
37. Muller P., Jesnowski R., Karle P., Renz R., Saller R., Stein H., Puschel K., von Rombs K., Nizze H., Liebe S., Wagner T., Gunzburg W. H. et al. Injection of encapsulated cells producing an ifosfamide-activating cytochrome P450 for targeted chemotherapy to pancreatic tumors, Ann N Y Acad. Sci. 880: 337-351, 1999.
38. Niculescu-Duvaz, I., Springer, C. J. (1997) Gene-directed enzyme prodrug therapy: a review of enzyme/prodrug combinations. Exp Opin Invest Drugs, 6, 685-703. Rigg, A., Sikora, K. (1997) Genetic prodrug activation therapy. Molecular Medicine Today, 359-366.
39. Nunez G., Benedict M. A., Hu Y. and Inohara N. Caspases: the proteases of the apoptotic pathway, Oncogene. 17: 3237-3245, 1998.
40. Ozols R. F., Louie K. G., Plowman J., Behrens B. C., Fine R. L., Dykes D. and Hamilton T. C. Enhanced melphalan cytotoxicity in human ovarian cancer in vitro and in tumor-bearing nude mice by buthionine sulfoximine depletion of glutathione, Biochem Pharmacol. 36: 147-153, 1987.
41. Pear W. S., Nolan G. P., Scott M. L. and Baltimore D. Production of high-titer helper-free retroviruses by transient transfection, Proc Natl Acad Sci USA. 90: 8392-8396, 1993.
42. Reed J. C. Bcl-2 Family Proteins: Relative Importance as Determinants of Chemoresistance in Cancer. In: J. A. Hickman and C. Dive (eds.), Apoptosis and Cancer Chemotherapy, 5 edition, pp. 99-116. Totowa, N.J.: Humana Press, 1999.
43. Rizk N. P., Chang M. Y., E1 Kouri C., Seth P., Kaiser L. R., Albelda S. M. and Amin K. M. The evaluation of adenoviral p53-mediated bystander effect in gene therapy of cancer, Cancer Gene Ther. 6: 291-301, 1999.
44. Roy N., Deveraux Q. L., Takahashi R., Salvesen G. S. and Reed J. C. The c-IAP-1 and c-IAP-2 proteins are direct inhibitors of specific caspases, Embo J. 16: 6914-6925, 1997.
45. Roy P., Yu L. J., Crespi C. L. and Waxman D. J. Development of a substrate-activity based approach to identify the major human liver P-450 catalysts of cyclophosphamide and ifosfamide activation based on cDNA-expressed activities and liver microsomal P-450 profiles, Drug Metab Dispos. 27: 655-666, 1999.
46. Russo J. E., Hilton J. and Colvin 0. M. The role of aldehyde dehydrogenase isozymes in cellular resistance to the alkylating agent cyclophosphamide, Prog Clin Biol Res. 290: 65-79, 1989.
47. Sahovic E. A., Colvin M., Hilton J. and Ogawa M. Role for aldehyde dehydrogenase in survival of progenitors for murine blast cell colonies after treatment with 4-hydroperoxycyclophosphamide in vitro, Cancer Res. 48: 1223-1226, 1988.
48. Scaffidi C., Schmitz I., Zha J., Korsmeyer S. J., Krammer P. H. and Peter M. E. Differential modulation of apoptosis sensitivity in CD95 type I and type II cells, J Biol. Chem. 274: 22532-22538, 1999.
49. Schwartz P. S. and Waxman D. J. Cyclophosphamide Induces Caspase 9-Dependent Apoptosis in 9L Tumor Cells, Mol Pharmacol. 60: 1268-1279, 2001.
50. SladekN. E. Metabolism of oxazaphosphorines, Pharmacol Ther. 37: 301-355, 1988.
51. Sladek N. E. Aldehyde dehydrogenase-mediated cellular relative insensitivity to the oxazaphosphorines, Curr Pharm Des. 5: 607-625., 1999.
52. Slee E. A., Harte M. T., Kluck R. M., Wolf B. B., Casiano C. A., Newmeyer D. D., Wang H. G., Reed J. C., Nicholson D. W., Alnemri E. S., Green D. R. and Martin S. J. Ordering the cytochrome c-initiated caspase cascade: hierarchical activation of caspases-2, -3, -6, -7, -8, and -10 in a caspase-9-dependent manner, J. Cell Biol. 144: 281-292, 1999.
53. Springer C. J. and Niculescu-Duvaz I. Prodrug-activating systems in suicide gene therapy, J Clin Invest. 105: 1161-1167, 2000.
54. Sun X. M., MacFarlane M., Zhuang J., Wolf B. B., Green D. R. and Cohen G. M. Distinct caspase cascades are initiated in receptor-mediated and chemical-induced apoptosis, J Biol. Chem. 274: 5053-5060, 1999.
55. Susin S. A., Lorenzo H. K., Zamzami N., Marzo I., Brenner C., Larochette N., Prevost M. C., Alzari P. M. and Kroemer G. Mitochondrial release of caspase-2 and -9 during the apoptotic process, J Exp Med. 189: 381-394, 1999.
56. Susin S. A., Lorenzo H. K., Zamzami N., Marzo I., Snow B. E., Brothers G. M., Mangion J., Jacotot E., Costantini P., Loeffler M., Larochette N., Goodlett D. R. et al. Molecular characterization of mitochondrial apoptosis-inducing factor [see comments], Nature. 397: 441-446, 1999.
57. Talanian R. V., Quinlan C., Trautz S., Hackett M. C., Mankovich J. A., Banach D., Ghayur T., Brady K. D. and Wong W. W. Substrate specificities of caspase family proteases, J Biol. Chem. 272: 9677-9682, 1997.
58. Tamm, I., Wang, Y., Sausville, E., Scudiero, D. A., Vigna, N., Oltersdorf, T. and Reed, J. C. (1998) IAP-family protein survivin inhibits caspase activity and apoptosis induced by Fas (CD95), Bax, caspases, and anticancer drugs. Cancer Res, 58, 5315-5320.
59. Thomberry N. A., Rano T. A., Peterson E. P., Rasper D. M., Timkey T., Garcia-Calvo M., Houtzager V. M., Nordstrom P. A., Roy S., Vaillancourt J. P., Chapman K. T. and Nicholson D. W. A combinatorial approach defines specificities of members of the caspase family and granzyme B. Functional relationships established for key mediators of apoptosis, J Biol. Chem. 272: 17907-17911, 1997.
60. Verhagen A. M., Ekert P. G., Pakusch M., Silke J., Connolly L. M., Reid G. E., Moritz R. L., Simpson R. J. and Vaux D. L. Identification of DIABLO, a mammalian protein that promotes apoptosis by binding to and antagonizing IAP proteins, Cell. 102: 43-53,2000.
61. Waxman D. J. Glutathione S-transferases: role in alkylating agent resistance and possible target for modulation chemotherapy—a review, Cancer Res. 50: 6449-6454, 1990.
62. Waxman D. J., Chen L., Hecht J. E. and Jounaidi Y. Cytochrome P450-based cancer gene therapy: recent advances and future prospects, Drug Metab Rev. 31: 503-522, 1999.
63. Waxman D. J., Light D. R. and Walsh C. Chiral sulfoxidations catalyzed by rat liver cytochromes P-450, Biochemistry. 21: 2499-2507, 1982.
64. Xie X., Zhao X., Liu Y., Zhang J., Matusik R. J., Slawin K. M. and Spencer D. M. Adenovirus-mediated tissue-targeted expression of a caspase-9-based artificial death switch for the treatment of prostate cancer, Cancer Res. 61: 6795-6804, 2001.
65. Zornig M., Hueber A., Baum W. and Evan G. Apoptosis regulators and their role in tumorigenesis, Biochim Biophys Acta. 1551: F1-37, 2001.

The references cited herein and throughout the specification are herein incorporated by reference in their entirety.

What is claimed is:

1. A method of prolonging expression of a heterologous gene in a cell transduced with a vector encoding the heterologous gene comprising
a) transducing the cell with a vector encoding an apoptosis inhibiting agent.

2. The method of claim 1, wherein the heterologous gene encodes a prodrug activating enzyme.

3. The method of claim 2 further comprising contacting the transduced cell of step a) with a prodrug.

4. The method of claim 3, wherein the cell is a neoplastic cell.

5. The method of claim 4, wherein the apoptosis inhibiting agent is a caspase pathway inhibiting agent.

6. The method of claim 5, wherein the caspase pathway inhibiting agent is selected from the group consisting of p35, p49, CrmA, XIAP, hIAP1, hIAP2, Naip, Bruce, Survivin, pIAP, CiIAP, OpIAP/CpIAP/AcIAP, ASFIAP, DIAP1, DIAP2, CeIAP1, CeIAP2, SpIAP, ScIAP, Bcl-2, Bcl-XL, and McI-1.

7. The method of claim 3, wherein the prodrug activating enzyme is selected from the group consisting of cytochrome P450, NADPH-P450 reductase, thymidine kinase, cytosine deaminase, nitroreductase, thymidine phosphorylase, purine nucleoside phosphorylase, alkaline phosphatase, carboxypeptidase A, carboxypeptidase G2, linamarase, beta-lactamase, xanthine oxidase, guanine phosphoribosyl transferase (GPT), deoxycytidine kinase, uracil phosphoribosyltransferase, carboxylesterase, and folylpolyglutamate synthetase.

8. The method of claim 3, wherein the prodrug is selected from the group consisting of cyclophosphamide (CPA) and other P450 prodrugs including bioreductive agents activated by P450 and/or NADPH-P450 reductase; ganciclovir, acyclovir and their analogs; 5-fluorocytosine; CB 1954 and other aromatic nitro prodrugs; 5′-deoxy-5-fluorouridine; 6-methylpurine-2′-deoxynucleoside; etoposide phosphate; methotrexate-(phenyl)alanine; benzoic acid mustard-glucuronide; amygdalin; cephalosporin-mustard carbamate; xanthine; 6-thioxanthine; cytosine arabinoside; 5-fluorouracil; irinotecan (CPT-11); edatrexate.

9. The method of claim 3, wherein the prodrug activating enzyme is cytochrome P450 and the prodrug is cyclophosphamide or ifosfamide.

10. The method of claim 9, wherein the prodrug-activating cytochrome P450 enzyme is selected from the group consisting of CYP 1A1, 1A2, 1B1, 2A6, 2B1, 2B6, 2B11, 2C3, 2C5, 2C6, 2C7, 2C8, 2C9, 2C11, 2C8, 2C19, 3A1, 3A2, 3A3, 3A4, 3A5 and 3A7.

11. A method of increasing the concentration of a chemotherapeutic drug in, or in the vicinity of, a target cell in a mammal in need thereof comprising the steps of:
a) transducing the target cell with a first vector comprising a nucleic acid encoding a prodrug activating enzyme;
b) transducing the target cell with a second vector comprising a nucleic acid encoding an apoptosis inhibiting agent; and
c) Subjecting the mammal to a prodrug that is activated by the prodrug activating enzyme of step a).

12. The method of claim 11, wherein the target cell is a neoplastic cell.

13. A method of increasing the concentration of a chemotherapeutic drug in, or in the vicinity of, a target cell in a mammal in need thereof comprising the steps of:
a) transducing the target cell with a vector comprising a nucleic acid encoding a prodrug activating enzyme and a nucleic acid encoding an apoptosis inhibiting agent; and
b) subjecting the mammal to a prodrug that is activated by the prodrug activating enzyme of step a).

14. The method of claim 11 or claim 13, wherein the apoptosis inhibiting agent is expressed under control of a regulatable promoter.

15. The method of claim 11 or claim 13, wherein the vector comprising a nucleic acid encoding an apoptosis inhibiting agent further comprises a factor that promotes apoptosis expressed under control of a regulatable promoter.

16. The method of claim 15, wherein the factor that promotes apoptosis is selected from the group consisting of Smac/Diablo a caspase, p53, Bax, Bak, Bcl-Xs, Bad, Bik, Bid, apoptosis inducing factor, and anti-sense or siRNA directed against the apoptosis inhibiting agent, an IAP or other anti-apoptotic factor.

17. The method of claim 11, wherein the vector comprising a nucleic acid encoding an apoptosis inhibiting agent further comprises a death receptor ligand expressed under control of a regulatable promoter.

18. The method of claim 17, wherein the death receptor ligand is selected from the group consisting of TNFα, Trail and Fas ligand.

19. The method of claim 1, wherein the heterologous gene encodes a soluble, or secretable, therapeutic factor.

20. The method of claim 19, wherein the therapeutic factor has anti-angiogenic, cytotoxic or immune modulatory activity.

21. The method of claim 19, wherein the cell is a neoplastic cell.

22. The method of claim 20, wherein the factor is selected from the group consisting of endostatin, angiostatin, VEGF receptor antibody, VEGF receptor-derived ectodomain, 16 kd prolactin fragment, platelet factor 4 and antibody, anti-sense agents or siRNA directed against angiogenic factors, the tumor necrosis factor superfamily, including TNFα, Fas ligand and Trail, a cytokine, interferon α, interferon β and interleukin 12.

23. A method of increasing the concentration of a soluble or secretable therapeutic factor in a target cell, in a mammal in need thereof comprising the steps of:
a) transducing the target cell with a vector comprising a nucleic acid encoding a soluble, or secretable, factor and a nucleic acid encoding an apoptosis inhibiting agent.

24. A method of increasing the concentration of a soluble or secretable therapeutic factor in the vicinity of a target cell in a mammal in need thereof comprising the steps of:
a) transducing the target cell with a first vector comprising a nucleic acid encoding a soluble, or secretable, therapeutic factor; and
b) further transducing the target cell with a nucleic acid encoding an apoptosis inhibiting agent, wherein said nucleic acid encoding and apoptosis inhibiting agent can be present in the first vector or in a separate vector.

25. The method of claim 23, wherein the soluble, or secretable therapeutic factor has anti-angiogenic, cytotoxic or immune modulatory activity.

26. The method of claim 25, wherein the soluble or secretable therapeutic factor is selected from the group consisting of endostatin, angiostatin, VEGF receptor antibody, VEGF receptor-derived ectodomain, 16 kd prolactin fragment, platelet factor 4 and antibody or anti-sense agents or siRNA directed against angiogenic factors, the tumor necrosis factor superfamily, including TNFα, Fas ligand and Trail, a cytokine, interferon α, interferon β, and interleukin 12.

27. The method of claim 23, wherein the apoptosis inhibiting agent comprises a death receptor inhibitory molecule.

28. The method of claim 27, wherein the death receptor pathway inhibitory molecule is selected from the group consisting of Fas-associated death domain-like ice inhibitory proteins (vFLIPs and cFLIPs), death receptor decoy receptors (DcR's), and dominant-negative Fas-associated death domain proteins (FADDs).

29. A method for enforcing vector spread in a host containing a vector encoding an heterologous gene comprising administering a replicating vector encoding an apoptosis inhibiting agent.

30. A method for enforcing vector spread in a host containing a vector encoding an heterologous gene comprising administering a replicating vector encoding an apoptosis inhibiting agent.

31. The method of claim 13, wherein the apoptosis inhibiting agent is expressed under control of a regulatable promoter.

32. The method of claim 13, wherein the vector comprising a nucleic acid encoding an apoptosis inhibiting agent, further comprises a factor that promotes apoptosis expressed under control of a regulatable promoter.

33. The method of claim 13, wherein the vector comprising a nucleic acid encoding an apoptosis inhibiting agent further comprises a death receptor ligand expressed under control of a regulatable promoter.

34. The method of claim 20, wherein the cell is a neoplastic cell.

35. The method of claim 24, wherein the soluble, or secretable therapeutic factor has anti-angiogenic, cytotoxic or immune modulatory activity.

36. The method of claim 24, wherein the apoptosis inhibiting agent comprises a death receptor inhibitory molecule.
